(19) United States
(12) Patent Application Publication
(10) Pub. No.: US 2021/0308101 A1
(43) Pub. Date: Oct. 7, 2021

(54) METHODS OF TREATING FIBROMYALGIA

(71) Applicant: Aptinyx Inc., Evanston, IL (US)

(72) Inventor: Torsten M. Madsen, Evanston, IL (US)

(21) Appl. No.: 17/094,595

(22) Filed: Nov. 10, 2020

Related U.S. Application Data
(60) Provisional application No. 62/933,566, filed on Nov. 11, 2019.

(51) Int. Cl. A61K 31/407; A61K 9/48; A61P 25/04

(52) U.S. Cl. CPC A61K 31/407; A61P 25/04; A61K 9/4858; A61K 9/4866

(57) ABSTRACT

Provided herein are methods of treating fibromyalgia, in a patient by administering to the patient an effective amount of NYX-2925 or a pharmaceutically acceptable salt thereof.

This application claims priority to and the benefit of U.S. Provisional Patent Application No. 62/933,566, filed on Nov. 11, 2019; the contents of which are hereby incorporated by reference herein in their entirety.

### BACKGROUND

Glutamate is the major excitatory neurotransmitter in the central nervous system and acts through activation of glutamate receptors. A portion of the receptors bind preferentially to N-methyl-D-aspartate (NMDA), and are therefore, termed NMDARs. Unlike other glutamate receptors found in the brain, such as α-amino-3-hydroxy-5-methyl-4-isoxazolepropionic acid or kainic acid receptors, the NMDARs are unique in that they have distinct binding sites for both glutamate and glycine, and binding by both ligands is required for receptor activation. The NMDARs are implicated in a number of physiological and pathological processes, including anxiety, cognition, learning, stroke, schizophrenia, Parkinson's disease, and neuropathic pain.

Fibromyalgia is a chronic, debilitating disorder typified by widespread musculoskeletal pain, accompanied by symptoms of fatigue, affected sleep, memory issues, and mood disorders. It is estimated that fibromyalgia affects 2-8% of the American adult population. Women are more commonly diagnosed than men (female to male ratio is 2:1 based on newer diagnostic criteria) and prevalence increases with age. Fibromyalgia is considered by some researchers to be a condition that is largely mediated in the central nervous system given that fibromyalgia sufferers often present without a direct peripheral insult or injury. Since the 2011 revision of the IASP definition of neuropathic pain, fibromyalgia has been excluded from the diagnosis of neuropathic pain. More recent studies however found newer evidence of pathophysiology, including small fiber neuropathy in patients with fibromyalgia. This may challenge the existing consensus and have implications on future diagnosis and management of this condition. A recent expert review described fibromyalgia as the prototypical central chronic pain syndrome.

Individuals suffering from fibromyalgia presently have limited treatment options available. Current treatment options include pregabalin, a calcium channel alpha-2-delta subunit ligand, and two norepinephrine and serotonin re-uptake inhibitors, duloxetine and milnacipran. These therapies have shown limited efficacy in treating fibromyalgia symptoms. However, for a large proportion of patients, treatment is insufficient.

Research suggests that the chronic widespread pain seen in fibromyalgia patients has a neurogenic origin. Higher levels of the neurochemicals implicated in pain processing, including nerve growth factor, substance P, and brain-derived neurotrophic factor, are present in the cerebrospinal fluid (CSF) of fibromyalgia patients when compared to healthy controls. In addition, Glu levels can be elevated in both the CSF and the brain of fibromyalgia patients. Glutamate may play a central role, by acting on NMDARs to increase the central amplification of pain perception, which is thought to manifest as allodynia and hyperalgesia in fibromyalgia patients. Thus, NMDARs are an attractive target for fibromyalgia therapeutic drug development.

As in fibromyalgia, the CNS modulates the experience of pain in people with neuropathic pain, with the rostroventromedial medulla, the dorsal anterior cingulate cortex (dACC), the insula, and other brain regions all thought to be involved. Neuropathic pain is caused by disease or injury of the somatosensory system as opposed to nociceptive pain where the sensory system is physiologically normal. While neuropathic pain can initially arise within the central or the peripheral nervous system through a wide range of etiologies, CNS modulation of the experience of pain is common, regardless of the specific precipitating factors or initial location of the pain.

A need continues to exist in the art for novel and more specific and/or potent compounds that are capable of treating fibromyalgia and its symptoms

### SUMMARY

In one aspect, provided herein are methods of treating fibromyalgia, in a patient in need thereof, comprising administering to the patient a therapeutically effective amount of (2S, 3R)-3-hydroxy-2-((R)-5-isobutyryl-1-oxo-2,5-diazaspiro[3.4]octan-2-yl)butanamide (“NYX-2925”), or a pharmaceutically acceptable salt thereof. In some embodiments, administering is administering orally. In some embodiments, administering is administering once daily.

### BRIEF DESCRIPTION OF FIGURES

FIG. 1A depicts a timeline of events in an exploratory study of patients treated with NYX-2925.

FIG. 1B is an example of a visual stimuli, where s=seconds.

FIG. 2A is a graph of change in dorsal anterior cingulate cortex (a pronociceptive brain region) resting Glx/tCr (glutamate & glutamine ratio/total creatine, a marker of excitatory neural activity) levels for 11 individual patients comparing the effect of a dose of placebo with the effect of a 20 mg dose of NYX-2925.

FIG. 2B is a graph of change in dorsal anterior cingulate cortex resting Glx levels for 11 individual patients comparing the effect of a dose of placebo with the effect of a 20 mg dose of NYX-2925.

FIG. 2C is a graph of change in dorsal anterior cingulate cortex resting Glx/tCr levels for 20 individual patients comparing the effect of a dose of placebo with the effect of a 20 mg dose of NYX-2925 (p=0.039).

FIG. 3A is a graph of change in posterior insula pain-related Glx/tCr levels for 11 individual patients comparing the effect of a dose of placebo with the effect of a 20 mg dose of NYX-2925.

FIG. 3B is a graph of change in posterior insula pain-related Glx/tCr levels for 11 individual patients comparing the effect of a dose of placebo with the effect of a 200 mg dose of NYX-2925.

FIG. 4A depicts brain images showing pain-evoked brain activity post placebo in subject 1.

FIG. 4B depicts brain images showing pain-evoked brain activity post 200 mg dose of NYX-2925 in subject 1.

FIG. 4C depicts brain images showing pain-evoked brain activity post placebo in subject 2.

FIG. 4D depicts brain images showing pain-evoked brain activity post 200 mg dose of NYX-2925 in subject 2.

FIG. 5 is a graph of change in posterior insula resting Glx/tCr levels for 19 individual patients comparing the effect of a dose of placebo with the effect of a 20 mg dose of NYX-2925 (p=0.032).

FIG. 6 change in pressure pain threshold (kg/cm²) for NYX-2925 (20 mg.) minus placebo versus the change in posterior insula Glx concentration AIU (post minus pre-pain) placebo visit (r=0.58; p=0.01).

FIG. 7 is a graph of change in dorsal anterior cingulate cortex resting Glx/tCr levels to LPreMotor/S1 for 17 individual patients comparing the effect of a dose of placebo with the effect of a 20 mg dose of NYX-2925 (P_{FWE}=0.030).

FIG. 8 is bar graph showing the mean change from baseline for average daily pain for placebo (Week 2); NYX-2925, 20 mg. QD (Week 4); NYX-2925, 200 mg. QD (Week 6); and Follow-Up, where for Weeks 2, 4, and 6: p<0.05 vs. baseline (BL); and for Week 6: p<0.05 vs. placebo.

FIG. 9 is bar graph showing the mean change from baseline for worse daily pain for placebo (Week 2); NYX-2925, 20 mg. QD (Week 4); NYX-2925, 200 mg. QD (Week 6); and Follow-Up, where for Week 6: p<0.05 vs. baseline (BL); and p<0.05 vs. placebo.

FIG. 10 is bar graph showing the mean change from baseline for total FIQR score for placebo (Week 2); NYX-2925, 20 mg. QD (Week 4); NYX-2925, 200 mg. QD (Week 6); and Follow-Up, where for Week 6: p<0.05 vs. baseline (BL); and p<0.05 vs. placebo.

FIG. 11 is bar graph showing the mean change from baseline for PROMIS_{FM} total fatigue profile score for placebo (Week 2); NYX-2925, 20 mg. QD (Week 4); NYX-2925, 200 mg. QD (Week 6); and Follow-Up, where for Week 6: p<0.05 vs. baseline (BL); and p<0.05 vs. placebo.

FIG. 12 is a graph showing change in dorsal anterior cingulate cortex glutamate plus glutamine is related to change in overall cuff pain unpleasantness (NYX-2925 20 mg Minus Placebo).

FIG. 13 is a graph showing placebo levels of dorsal anterior cingulate cortex glutamate plus glutamine total creatine are correlated with subsequent changes in cuff pressure pain unpleasantness (20 mg Drug Minus Placebo).

FIG. 14A is a graph showing correlation between combined glutamate and glutamine levels on composite sensory testing after 6 weeks of treatment (NYX-2925 200 mg).

FIG. 14B is a graph showing correlation between combined glutamate and glutamine levels on patient-reported outcome of pain, fatigue, sleep, anxiety, and depression after 6 weeks of treatment (NYX-2925 200 mg).

FIG. 15 is a bar graph showing change in posterior insula combined glutamate plus glutamine metabolites to creatine ratio for placebo and NYX-2925 20 mg and 200 mg.

FIG. 16 is a graph showing the relationship between pressure pain and combined glutamate plus glutamine metabolites concentration at week 2 (placebo).

FIG. 17 is a graph showing the relationship between pressure pain and combined glutamate plus glutamine metabolites concentration at week 6 (NYX-2925 200 mg).

FIG. 18A is a graph showing change in pressure pain threshold for 20 mg NYX-2925 minus placebo versus change in posterior insula glutamate plus glutamine baseline.

FIG. 18B is a graph showing change in pressure pain threshold for 20 mg NYX-2925 minus placebo versus change in posterior insula glutamate plus glutamine placebo.

FIG. 19 is a graph showing decreased pain revoked release of posterior insula combined glutamate and glutamine metabolites on NYX-2925 200 mg is associated with reductions in pain severity from placebo.

FIG. 20 is a graph showing correlation between dorsal anterior cingulate cortex and right precuneus connectivity with brief pain interference scores for NYX-2925 20 mg versus placebo.

FIG. 21 is a graph showing correlation between perigenual anterior cingulate cortex and left precuneus connectivity with ratings of MAST testing unpleasantness for NYX-2925 20 mg versus placebo.

FIG. 22 is a graph showing correlation between medial prefrontal cortex and mid cingulate cortex connectivity with brief pain inventory interference scores for NYX-2925 200 mg versus placebo.

FIG. 23 is a graph showing correlation between medial prefrontal cortex and mid cingulate cortex connectivity with brief pain inventory interference scores for NYX-2925 200 mg versus placebo.

FIG. 24 is a graph showing correlation between thalamus and right/left cuneus connectivity with multi-modal automated sensory testing mean pain intensity ratings for NYX-2925 200 mg versus baseline.

FIG. 25 is a graph showing correlation between thalamus and right/left cuneus connectivity with multi-modal automated sensory testing pain 50 thresholds for NYX-2925 200 mg versus baseline.

### DETAILED DESCRIPTION

As generally described herein, the present disclosure provides methods of treating fibromyalgia, using a compound of the disclosure, namely, (2S, 3R)-3-hydroxy-2-((R)-5-isobutyryl-1-oxo-2,5-diazaspiro[3.4]octan-2-yl)butanamide (“NYX-2925”), or a pharmaceutically acceptable salt thereof.

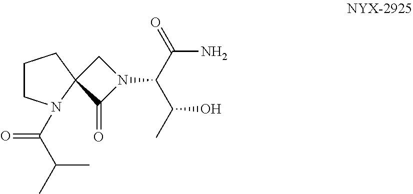 [SMILES: CC(C)C(=O)N1CCC[C@]12CN([C@H](C(N)=O)[C@@H](C)O)C2=O]

NYX-2925 is an N-methyl-D-aspartate receptor (NMDAR) modulator, and at low concentrations of endogenous agonist (glycine or D-serine) and in the presence of glutamate, NYX-2925 partially activates the NMDAR. NYX-2925 appears to act at a binding site that is distinct from NMDAR agonists or antagonists studied to date, such as D-cycloserine, ketamine, MK-801, or kynurenic acid. The mode of action of NYX-2925 seems to be distinct from that of all existing and emerging drugs that are indicated for the treatment of neuropathic pain. While current medications target individual elements of pain signal transmission or modulation, NYX-2925 can modulate multiple synaptic relays within pain circuits.

### Definitions

To facilitate an understanding of the present invention, a number of terms and phrases are defined below.

List of Abbreviations:

| | |
|---|---|
| ¹H-MRS | Proton Magnetic Resonance Spectroscopy |
| ACR | American College of Rheumatology |
| AE | Adverse Event |
| BOLD | Blood Oxygenation Level Dependent |
| BPI | Brief Pain Inventory |
| CPFRC | Chronic Pain and Fatigue Research Center |
| CRF | Case Report Form |
| CSF | Cerebrospinal Fluid |
| C-SSRS | Columbia-Suicide Severity Rating Scale |
| DAN | Dorsal Attention Network |
| DMN | Default Mode Network |
| ECG | Electrocardiogram |
| eCRF | Electronic Case Report Form |
| fcMRI | Functional Connectivity Magnetic Resonance Imaging |
| FDA | Food and Drug Administration |
| FDR | False Discovery Rate |
| FIQR | Revised Fibromyalgia Impact Questionnaire |
| fMRI | Functional Magnetic Resonance Imaging |
| FMRIB | Functional Magnetic Resonance Imaging of the Brain |
| FSL | FMRIB Software Library |
| FSQ | Fibromyalgia Survey Questionnaire |
| FWE | Family-wise Error |
| FWHM | Full Width at Half Maximum |
| GIFT | Group ICA of Fmri Toolbox |
| GLM | General Linear Model |
| Glu | Glutamate |
| Glx | Glutamate plus Glutamine |
| HADS | Hospital Anxiety and Depression Scale |
| HIV | Human Immunodeficiency Virus |
| ICs | Independent Components |
| ICA | Independent Component Analysis |
| MAST | Multi-Modal Automated Sensory Testing |
| MATLAB | Matrix Laboratory |
| MISCI | Multidimensional Inventory of Subject Cognitive |
| | Impairment |
| MNI | Montreal Neurological Institute |
| MR | Magnetic Resonance |
| MRI | Magnetic Resonance Imaging |
| N/A | Not Applicable |
| NPRS | Numeric Pain Rating Scale |
| NYX-2925 | Aptinyx's Investigational Study Drug |
| OREs | Other Reportable Events |
| PGI-C | Patient Global Impression of Change |
| PO | Per Oral |
| PROMIS | Patient Reported Outcomes Measurement Information |
| | System |
| PROMIS_{FM} | Patient Reported Outcomes Measurement Information |
| | System Fibromyalgia |
| QD | Once Daily |
| QST | Quantitative Sensory Testing |
| rs-fcMRI | Resting State Functional Connectivity Magnetic Resonance |
| | Imaging |
| SAP | Statistical Analysis Plan |
| SLN | Salience Network |
| SPM | Statistical Parametric Mapping |
| SPM12 | Statistical Parametric Mapping version 12 |
| SPSS | Statistical Package for the Social Sciences |
| WPI | Widespread Pain Index |

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where formulations and kits are described as having, including, or comprising specific components, or where processes and methods are described as having, including, or comprising specific steps, it is contemplated that, additionally, there are formulation sand kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a formulation or a method described herein can be combined in a variety of ways without departing from the spirit and scope of the present invention, whether explicit or implicit herein. For example, where reference is made to a particular compound, that compound can be used in various embodiments of formulations of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred from the context.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, formulations specifying a percentage are by weight unless otherwise specified. Further, if a variable is not accompanied by a definition, then the previous definition of the variable controls.

As used herein, “pharmaceutical composition” or “pharmaceutical formulation” refers to the combination of a therapeutically active agent with a pharmaceutically acceptable excipient, making the composition especially suitable for diagnostic or therapeutic use in vivo or ex vivo.

“Pharmaceutically acceptable” refers to compounds, molecular entities, compositions, materials and/or dosage forms that do not produce an adverse, allergic or other untoward reaction when administered to an animal, or a human, as appropriate, and/or that are approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to any salt of an acidic or a basic group that is present in a compound of the present invention (e.g., NYX-2925), which salt is compatible with pharmaceutical administration.

As is known to those of skill in the art, “salts” of compounds may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium and potassium) hydroxides, alkaline earth metal (e.g., magnesium and calcium) hydroxides, ammonia, and compounds of formula NW₄⁺, wherein W is C₁₋₄ alkyl, and the like.

Examples of salts include, but are not limited, to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, K⁺, Ca²⁺, NH₄⁺, and NW₄⁺ (where W can be a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, “pharmaceutically acceptable excipient” refers to a substance that aids the administration of an active agent to and/or absorption by a subject and can be included in the compositions or formulations of the present invention without causing a significant adverse toxicological effect on the patient. Non-limiting examples of pharmaceutically acceptable excipients include water, NaCl, normal saline solutions, such as a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. For examples of excipients, see Martin, Remington's Pharmaceutical Sciences, 15th Ed., Mack Publ. Co., Easton, Pa. (1975).

The term “AUC” refers to the area under the time/plasma concentration curve after administration of the pharmaceutical formulation. AUC_{0-infinity} denotes the area under the plasma concentration versus time curve from time 0 to infinity; AUC₀₋ₜ denotes the area under the plasma concentration versus time curve from time 0 to time t. It should be appreciated that AUC values can be determined by known methods in the art.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

The term “Cₘₐₓ” refers to the maximum concentration of a therapeutic agent (e.g., NYX-2925) in the blood (e.g., plasma) following administration of the pharmaceutical formulation.

The term “tₘₐₓ” refers to the time in hours when Cₘₐₓ is achieved following administration of the pharmaceutical formulation comprising a therapeutic agent (e.g., NYX-2925).

As used herein, “solid dosage form” means a pharmaceutical dose(s) in solid form, e.g., tablets, capsules, granules, powders, sachets, reconstitutable powders, dry powder inhalers and chewables.

By “co-administer” it is meant that a formulation described herein is administered at the same time, just prior to, or just after the administration of one or more additional therapies (e.g., analgesic, anti-cancer agent, chemotherapeutic, or treatment for a neurodegenerative disease). NYX-2925, or a pharmaceutically acceptable salt thereof, can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (e.g., “therapeutic treatment”).

In general, an “effective amount” or “therapeutically effective amount” of a compound or a pharmaceutical formulation refers to an amount sufficient to elicit the desired biological response, e.g., to treat fibromyalgia. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject.

As used herein, “PBO” is placebo.

As used herein, “MMRM” is mixed-effect model repeated measure.

As used herein, “fMRI” is Functional Magnetic Resonance Imaging.” Functional magnetic resonance imaging is a noninvasive technique for measuring brain activity by distinguishing changes in blood oxygenation and flow which occur in response to neural activity. Active sections of the brain consume oxygen, therefore requiring increased blood flow. The fMRI detects those hemodynamic changes and provides activation maps, which show the sections of the brain involved in a specific physiological process.

This study used a harmonized Human Connectome Protocol (HCP), adapted from the Adolescent Brain Cognitive Development (ABCD) protocol, optimized for quantitative accuracy and consistency of imaging biomarkers across scanners and time. This was based off the multisite ABCD study, which is already implementing a cross-platform harmonized protocol based off the Lifespan HCP. During the initial development phase of both sequences and tasks, parameters were harmonized, within the range of recommended HCP protocols, as informed by then current HCP, Lifespan Connectome, and ABCD project knowledge.

Crossover testing of subjects at both sites was used to assure MRI equivalence across sites, as well as behavioral/performance measures and overall methodology. Phantom Quality Assurance scans were also performed to ensure ongoing equipment/sequence matching based on quality assurance protocols from the Federal Biomedical Informatics Research Network project and the ABCD project. This included standard and Federal Biomedical Informatics Research Network quality assurance scans, as well as multiband fMRI scans, to give measures that included spatial and temporal signal-to-noise ratio and signal-to-fluctuation-noise ratio, signal stability, image distortion, ghosting level, and others. This allowed sites to actively monitor and correct for any transient equipment artifacts (such as spike noise or bad coil channels) as well as gradual system-wide degradation (such as eddy currents and magnetic field inhomogeneity) during the course of this study.

Unless otherwise specified, the endpoints of interest for both pharmacodynamic and exploratory efficacy analysis were as follows:
Placebo at Week 2 Change from Baseline (neuroimaging and QST only)
NYX-2925 20 mg PO QD at Week 4 Change from Baseline
NYX-2925 20 mg PO QD at Week 4 Change from Week 2 (Placebo)
NYX-2925 200 mg PO QD at Week 6 Change from Baseline
NYX-2925 200 mg PO QD at Week 6 Change from Week 2 (Placebo)
NYX-2925 200 mg PO QD at Week 6 Change from Week 4 (NYX-2925 20 mg PO QD)
Follow-Up at Week 7 Change from Week 6 (NYX-2925 200 mg PO QD)

When reporting pharmacodynamic results, nominal p-values for comparisons are described as statistically significant when p<0.05. In those instances where 0.05<p<0.10, the observed differences are described as marginal. For comparisons wherein p>0.10, the observations are noted as not statistically significant.

Single subject fMRI data were entered into various paired sample t-tests. The endpoints of interest were the change in brain metabolite levels, activation patterns, and connectivity at the time points identified above.

Statistical effects were significant at voxel-wise threshold of p<0.001 and p<0.05 cluster-corrected (either false discovery rate or family-wise error corrected). Analyses were also performed with a series of liberal voxel-wise p-value thresholds (p<0.01 to <0.001) to assess effects of smaller magnitude. Data with significant measurement artifacts, including uncorrectable/excessive head motion for fMRI data and Cramer-Rao bounds >20% for ¹H-MRS metabolite concentrations, were set to missing for all analyses. The primary analyses for ¹H-MRS (posterior insula and anterior cingulate Glx levels) were paired t-tests and/or nonparametric Wilcoxon signed-rank tests between placebo images versus NYX-2925 (20 mg or 200 mg).

Patient-reported symptom data collected during the neuroimaging procedure, including NRS scores of clinical pain and evoked stimulus intensity and unpleasantness, were treated as neuroimaging outcome covariates in post hoc exploratory analyses.

As used herein, “fcMRI” is Functional Connectivity Magnetic Resonance Imaging.” fcMRI investigations are conducted with subjects simply resting in the scanner. These data can then be analyzed with techniques such as independent component analysis (ICA) and seed-based voxel connectivity. While multiple resting state (rs) networks have been shown to be altered in pain states, this analysis focused on three cardinal networks known to be related to pain and sensory processing: default mode network (DMN), the dorsal attention network (DAN), and the salience network (SLN).

Resting state functional connectivity magnetic resonance imaging (rs-fcMRI) also uses noninvasive techniques to map blood flow in the brain. In rs-fcMRI, neural connections between different areas of the brain are identified by mapping regions of synchronous blood flow fluctuations. Synchronous blood fluctuations are examined to determine the strength of neural connections between different brain regions.

Resting 6-minute scans, with subjects resting comfortably in the scanner with eyes open and fixated on a crosshair, were completed at the beginning of the scan session. Whole brain BOLD rs-fcMRI data were collected following same parameters used for evoked fMRI. Physiological data were also collected simultaneously with rs-fcMRI data, as cardiorespiratory fluctuations are known to influence rs-fcMRI intrinsic connectivity estimation within several brain networks

In order to quantify intrinsic brain connectivity, rs-fcMRI data were analyzed with both dual-regression ICA and seed-voxel approaches. These approaches are complementary, as they quantify intrinsic brain connectivity on a network level (dual regression probabilistic ICA) and a more specific region-focused level (seed-voxel).

While multiple resting state networks have been shown to be altered in pain states, this analysis focused on 3 cardinal networks known to be related to pain and sensory processing: Default Mode Network, Dorsal Attention Network, and Salience Network.

Resting state functional connectivity MRI data were preprocessed and analyzed using statistical parametric mapping software package Version 12, the CONN functional connectivity toolbox, and the GIFT toolbar, all running on MATLAB Version 9.2 or later. Upon collection of rs-fcMRI data, physiological artifacts were first removed using a custom MATLAB algorithm and slice-time corrected using Functional Magnetic Resonance Imaging of the Brain Software Library software. Preprocessing steps included motion correction, realignment, registration, normalization to standard Montreal Neurological Institute template, and smoothing (full width at half maximum Gaussian kernel of 6 or 8 mm).

Seed to whole brain functional connectivity analysis was performed using the CONN toolbox, Version 15, running on MATLAB 7.5b. Seed regions were first identified using previously published fMRI studies within chronic pain, including: regions within the Salience Network such as the insula and Default Mode Network regions, including the posterior cingulate, precuneus, and inferior parietal lobule. Spheres were created (5 mm radius) around peak voxel coordinates and white matter, CSF, and motion parameters were used as covariates of no interest. Seed-based analyses were also performed using regions of interest assessed with single voxel ¹H-MRS regions showing NYX-2925 effects on Glx level. Data were band pass filtered (0.01 to 0.1 Hz) to remove linear drifts and high frequency noise. First level analysis correlated the time course from the seed to all brain voxels, creating connectivity maps for each seed region. These connectivity maps were passed up to group-level analyses comparing differences in connectivity at the visits of interest in statistical parametric mapping.

Group ICA was performed using the GIFT toolbar and component estimates were validated using Icasso software for 10 iterations. The number of independent components was limited to 25 to minimize splitting into subcomponents. Subject-specific spatial maps and time courses were back reconstructed using spatiotemporal regression and dual regression in GIFT. As for seed-based analyses, individual maps were passed onto group second level analyses in statistical parametric mapping, where differences in resting state network connectivity between placebo and drug images were performed.

As used herein, “Functional Magnetic Resonance Imaging of Evoked Pain and Visual Stimulation” refers to the following procedure. During evoked pain and visual stimulation fMRI paradigms, participants were lying in the scanner as they received either pressure stimulation to their calf muscle or they experienced an unpleasant visual stimulus. For both pain and visual stimulation, whole-brain Blood Oxygenation Level Dependent (BOLD) functional images were acquired.

Each subject underwent a 12-minute block design scan, during which 3 cuff pressure levels (2 equal pressures=15 to 30 and 100 to 115 mmHg; and 1 equal pain=40 to 60/100 numerical rating scale [NRS] units) were applied to the left calf 3 to 6 times each using the pressure cuff device in pseudo-random order for a total of 12 pressure stimulations. Pressure levels for each subject were determined during the QST assessment. Each pressure stimulus was applied for 14 seconds. Prior to each pressure stimulus, a 4-second visual cue was presented to signal upcoming cuff pressure to limit any stimulus-onset startle reflex. Subjects' head motion was minimized using foam pads around the head, and a strap secured across the forehead.

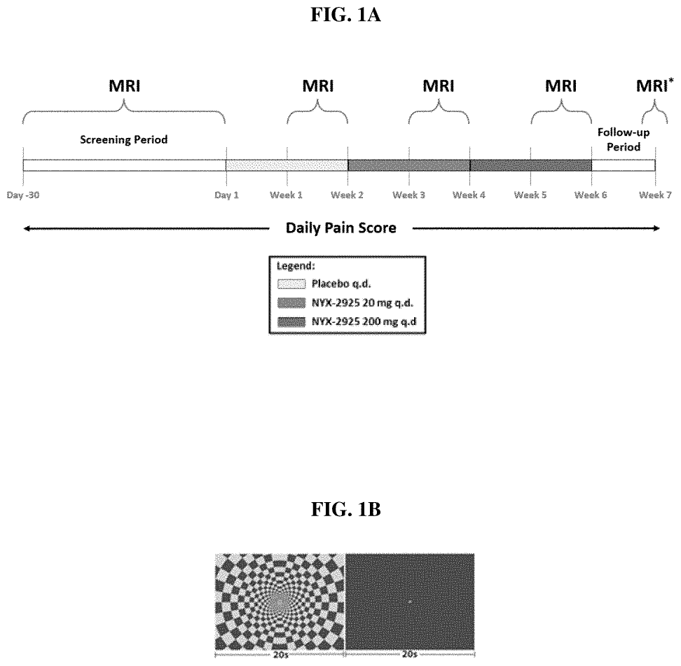

In addition to hypersensitivity to painful stimuli applied to the body, many chronic pain subjects report hypersensitivity to nonpainful sensory stimuli, including auditory, olfactory, and visual stimulation, suggesting a global state of multisensory amplification in centralized pain. Neural mechanisms of this multimodal hypersensitivity were assessed using fMRI. Whole brain BOLD functional images were acquired. The visual stimulation task was 3 minutes in duration and consisted of 2 different visual stimuli presented in a block design. The control stimulus was a fixed crosshair centered in the middle of a solid color background, whereas the experimental stimulus was a flashing annulus checkerboard (8 Hz frequency) (see below figure of example of visual stimuli). These 2 stimuli were interleaved into independent 20-second blocks throughout the duration of the task. During fMRI visual stimulation, the luminance remained fixed. Subjects were asked to keep their eyes open and remain focused on the display on the screen. See, e.g., FIG. 1B.

As with functional connectivity analyses, pre-processing of images occurs using SPM12. The first 5 images were discarded from the data set and not used for further analysis in order to correct for equilibration effects. Pre-processing steps included: physio-correction, motion correction, coregistration, normalization, and smoothing (FWHM 6 or 8 mm). First level analyses were performed using the general linear model (GLM) implemented in SPM12. Motion parameters from re-alignment were modelled as regressors of no interest. Pain and visual blocks were convolved with a canonical hemodynamic response function and contrasted with rest blocks of no stimulation. These activation maps were then passed up to group-level analyses comparing differences in activations at the analysis visits of interest in SPM. Resulting maps were set at an uncorrected voxel threshold of p<0.001, with p<0.05 FDR or FWE significance corrected for multiple comparisons. The primary analyses were a paired sample t-tests between placebo images and NYX-2925 images (either 20 or 200 mg).

As used herein, “¹H-MRS” is Magnetic Resonance Spectroscopy (1H-MRS) or Proton Magnetic Resonance Spectroscopy. Proton magnetic resonance spectroscopy is a noninvasive imaging technique that allows in vivo determination of biochemical parameters within brain tissue. Proton magnetic resonance spectroscopy data were used to collect brain neurotransmitter levels of Glu and combined glutamate+glutamine metabolites (Glx). This method provides metrics amenable to longitudinal studies such as those proposed here. High-resolution anatomical scans can isolate identical brain structures within individuals over time, thus minimizing error that otherwise would occur because of slight differences in voxel location from 1 evaluation to the next. Previous ¹H-MRS studies have utilized this approach to examine changes in the levels of CNS metabolites in test-retest studies of drug effects. This study expanded the investigation to a variety of regions throughout the brain including:
Right anterior insula
Right posterior insula (This region was acquired twice: once prior to and once following evoked cuff pain. All other voxels were acquired once.)
Perigenual/rostral anterior cingulate cortex (midline)
Dorsal/anterior cingulate cortex (midline)
Precuneus (midline)
Medial prefrontal cortex
Thalamus (optional if time permitted)

Proton magnetic resonance spectroscopy data were collected using the same scanners used for fMRI. Voxels of interest were placed in each of the aforementioned brain regions to obtain levels of Glu and Glx.

The MRI sessions required approximately 90 minutes to complete. Clinical and evoked pain ratings were assessed at several times, including immediately before, during, and immediately after the MRI session using 0 to 100 NRS (0=“no pain” and 100=“most intense pain imaginable”).

Raw data from each single-voxel spectroscopy sequence underwent manual post-processing using ¹H-MRS software (LCModel; Stephen Provencher, Oakville, Ontario, Canada). LCModel uses a linear combination of individual spectra obtained from pure molecular species to fit the experimental spectra. Glx values were calculated as an absolute concentration using the water signal for normalization or as a ratio to creatine. Correction for cerebral spinal fluid (CSF) volume, which can dilute ¹H-MRS-derived Glx values, was also performed. For this we used Voxel Based Morphometry, a toolbox that operates within SPM. Metabolite concentrations were excluded if the Cramér-Rao bounds were greater than 20%.

The analysis visit comparisons stated were analyzed using paired t-tests and/or nonparametric Wilcoxon signed-rank tests of Glx levels at each voxel independently in SPSS. The primary analyses were comparisons between posterior insula and anterior cingulate cortex Glx levels following placebo and NYX-2925 (either 20 or 200 mg) administration.

Other efficacy assessments, as used herein, include “Multi-Modal Automated Sensory Testing” (MAST) Pressure Pain Sensitivity, “Cuff Algometry,” and “Visual Stimulation Task.”

The multi-modal automated sensory testing (MAST) system was used to measure localized pressure sensitivity at an asymptomatic, neutral body site, the thumbnail. The MAST system is a nonsignificant-risk investigational device that applies a computer-controlled mechanical stimulation at precisely controlled intensity levels and durations. The validity in the measurement of centrally-mediated pain sensitivity has been discussed extensively. The MAST system delivered an ascending series of computer-controlled, 5-second pressures at 20-second intervals (with 20% jitter) to the subject's thumbnail bed, beginning at 0.25 to 0.5 kg/cm² and increasing in steps of 0.25 to 0.50 kg/cm². Pain intensity was rated after each pressure on a 0 to 100 NRS. Testing was terminated when the first of 3 possible stop conditions were met: 1) subject reached her personal pain tolerance and requested to stop the test, 2) subject reported a pain intensity rating of >80, or 3) the maximum pressure of 10 kg/cm² was delivered. A regression model was used fit the stimulus-response data obtained from this procedure and interpolate Pain50, defined as the pressure intensity that evokes a moderate level of suprathreshold pressure pain (i.e., 50/100). Additional derived variables included: 1) Pressure pain threshold, defined as the first pressure in a series of at least 2 consecutive pressures that elicited a NRS pain rating >0; 2) slope of the stimulus-response function as a measure of the rate of pain increase; and 3) pressure pain tolerance, defined as the last pressure recorded in the stimulus response profile. These variables were used to determine the effect of drug and placebo treatments on pain sensitivity.

Localized pressure pain sensitivity was also measured bilaterally at 3 additional body regions: trapezius, wrist, and patella. Examination of multiple sites across all quadrants of the body permitted analysis of the distribution of pain sensitivity. Sensitivity was measured using a digital algometer with a 1-cm² flat rubber probe. At each site, pressure was manually increased at a steady rate of 30 to 50 kilopascal (1000 kilopascal maximum) until the subject indicated the first sensation of pain (i.e., the pressure pain threshold). Measurements were conducted 2 to 3 times (15- to 20-second intervals) at each site.

Large volume, deep tissue pressure sensitivity was assessed by cuff algometry. Calibrated cuff pain stimuli were delivered to the gastrocnemius area of the lower leg using a validated MRI-compatible cuff pain device. This system included an air compressor, computerized pressure controller, and a 13.5 cm×82.5 cm Velcro-adjusted pressure cuff (similar to a blood pressure cuff). Subjects first received an ascending series of cuff pressures, starting at 20 mmHg and increasing in 20-mmHg steps (10- to 14-second duration pressures, 20-second interstimulus interval) to tolerance or a maximum of 400 mmHg. Each pressure was rated after deflation on a 0 to 100 NRS. These pain ratings were used to interpolate a series of 9 tolerable cuff pressures that were delivered in pseudo-randomized order and rated individually. These data were used to generate stimulus response curves representing deep tissue sensitivity. Curves were constructed for each participant and used for analysis of drug and placebo effects. An abbreviated form of cuff algometry was performed during fMRI as an evoked pain stimulus.

To measure non-somatic sensitivity, a series of nonpainful, yet aversive, visual stimuli were presented to each subject. This 10-minute task consisted of 2 different visual stimuli presented in an alternating block design. The control stimulus was a solid color background, whereas experimental stimulus was a flashing annulus checkerboard. These 2 stimuli were interleaved into independent 8- to 30-second blocks throughout the duration of the task. Subjects viewed the stimuli on a calibrated liquid crystal display computer screen designed for high resolution image presentation. Stimuli were presented at varying brightness levels and/or color patterns, and each stimulus presentation was rated on scales of sensory intensity and unpleasantness. These data were to be used to generate stimulus-response curves depicting visual stimulus-evoked aversion and used for analysis. A redacted version of this task was administered during fMRI.

QST data were analyzed at the University of Michigan CPFRC. Dependent variables were the MAST derived outcomes (Pain50, pressure pain threshold, pain tolerance, and pain ratings at 2 kg/cm2), algometry-derived pressure pain thresholds (mean of three trials) at each anatomical site, average cuff intensity ratings (mean of ratings at all stimulus levels during the ascending or random sequences), and average visual task unpleasantness and brightness intensity ratings (mean of ratings at all stimulus levels). Practice data were not analyzed. Overall pain intensity and unpleasantness NRS scores as well as stimulus response curves for each test were assessed during post hoc analyses. Data points displaying measurement artefacts, including device malfunction or user error, were set to missing. Single subject QST variables were analyzed using paired t-tests and/or nonparametric Wilcoxon signed-rank tests in SPSS or R.

As used herein, “NPRS” is Numeric Pain Rating Scale Score. Numeric pain rating scale is a unidimensional segmented numeric version of the visual analog scale. The NPRS score assessing average pain intensity in the past 24 hours are based on an 11-point numerical rating scale/Likert scale ranging from 0 to 10. Where 0 represents no pain and 10 is the worst pain imaginable. Individual one-time scores were collected at the Screening and Day 1 visits on the CRF in order to confirm eligibility status for the subjects. These CRF pain scores were not used in the analysis tables. Instead, the pain scores collected by eRT via phone data collection were used in the analysis tables. eRT collected a variable if the call was completed per the requirements. In the event the subject did not complete the call (i.e. hung up the phone too early), any pain scores data collected were used for that study day if another call was not completed within that same study day. The daily pain scores were summarized using the mean and were calculated for the analysis visits at Baseline and Weeks 1-7. There needed to be at least 4 daily pain scores collected within a post-baseline analysis visit in order to calculate the mean pain score for that visit. Each mean analysis visit score took on values ranging from 0 to 10 and was rounded off by one number after the decimal point. No missing data imputations were used for these individual values or computed means. The actual values/means, changes from baseline, and changes from treatment periods at each analysis visit were summarized using descriptive statistics. Additionally, the treatment group comparisons were analyzed using the paired t-test and nonparametric Wilcoxon signed-rank test. All data collected and derived were presented in subject listings. The pain scores collected by eRT and the CRF were presented in separate subject listings. With the exception of placebo at Week 2, the exploratory endpoints of interest with this data were as described above.

The collection methods of the NPRS score assessing worst pain intensity in the past 24 hours were the same as the efficacy data for the NPRS score assessing average pain intensity in the past 24 hours and was analyzed using the same techniques. The worst pain score must be greater than or equal to the average pain score for each assessment. In the event a worst pain score was less than the average pain, both pain scores were removed from calculating the mean weekly analysis visit scores. These collected pain scores were presented in the subject listings with a note that they would be removed from the analysis tables.

As used herein, “Brief Pain Inventory” (BPI)—Short Form is a brief, simple patient-completed numeric rating scale that assesses the severity of pain (Severity scale), its impact on daily functioning (Interference scale), and other aspects of pain (e.g., location of pain, relief from medications). A short form version of the BPI, including the 4-item pain Severity scale (Worst Pain, Least Pain, Average Pain, and Pain Now) and the 7-item pain Interference scale (General Activity, Mood, Walking Ability, Normal Work, Relations with Other People, Sleep, Enjoyment of Life) were the main BPI-Short Form analyses. Each BPI item uses a 0 to 10 numeric rating scale anchored at zero for “no pain” and 10 for “pain as bad as you can imagine” for Severity, and “does not interfere” to “completely interferes” for Interference. If at least 3 of the 4 Severity items have a reported score, then the Pain Severity Index was the calculated mean. Otherwise, the Pain Severity Index was set to missing. If at least 4 of the 7 Interference items had a reported score, then the Pain Interference Index was the calculated mean. Otherwise, the Pain Interference Index was set to missing. No imputation methods for missing data were applied for this data. The Pain Severity Index and the Pain Interference Index were referred to as composite scores.

There may be dates in which worst pain in the last 24 hours has scores that are less than the least pain in the last 24 hours and/or right now pain scores. Also, the least pain in the last 24 hours scores may be greater than the worst pain in the last 24 hours and/or right now pain scores. This may be due to the collection of the data that cannot be changed due to the subject's confusion of the three scores. The Pain Severity Index score equally distributes all these three scores and the average pain score. Therefore, the Pain Severity Index score is not impacted by any of these scores being mixed up. As a result, the scores were analyzed based on the values collected.

The 2 composite scores took on values ranging from 0 to 10 and were rounded off by one number after the decimal point. A total pain score for BPI-Short Form was not calculated. The BPI-Short Form assessment was collected and analyzed at visits Screening, Day 1, Week 2, Week 4, Week 6, and Follow-up (Week 7). The observed composite scores, changes from baseline, and changes from treatment periods were summarized using descriptive statistics. Additionally, the treatment group comparisons were analyzed using the paired t-test and nonparametric Wilcoxon signed-rank test. Data collected during an Early Termination visit were not summarized in the tables. All BPI-Short Form data collected and the calculated composite scores at each visit were presented in a subject listing. With the exception of placebo at Week 2, the exploratory endpoints of interest with this data were as described above.
BPI-Short Form question 1, “Throughout our lives, most of us have had pain from time to time (such as minor headaches, sprains, and toothaches). Have you had pain other than these everyday kinds of pain today?” is a categorical response (yes/no). This data was presented in a subject listing, but not summarized in a table.
BPI-Short Form question 8, “In the last 24 hours, how much relief have pain treatments or medications provided? Please circle the one percentage that most shows how much relief you have received.” was collected on the CRF and had values that range from 0% (no relief) to 100% (complete relief). This question was included in the descriptive statistics, but was not analyzed using any paired t-test and nonparametric Wilcoxon signed-rank test comparisons.
BPI-Short Form question 2, “On the diagram, shade in the areas where you feel pain. Put an X on the area that hurts the most.” was not collected on the CRF.
BPI-Short Form question 7, “What treatments or medications are you receiving for your pain?” was provided in subject listings, but not in any summary tables.

As used herein, the “PROMIS_{FM}” fatigue profile is Patient Reported Outcomes Measurement Information System Fibromyalgia, which is a 16-question instrument which has four 4-item short forms that examines the intensity of the impact of fatigue in in social, cognitive and motivation subdomains. All 16 questions are based on a 5-point numerical rating scale/Likert scale ranging from 1 to 5, with different categorical responses. Lower scores represent a better quality of life. The PROMIS_{FM} fatigue profile is made of the 4 following subdomains:
Experience Subdomain Score
   3 Questions with Scores Ranging from 1 (Not at All) to 5 (Very Much):
How tired did you feel on average?
How fatigued were you on average?
How exhausted were you on average?
   1 Question with Scores Ranging from 1 (None) to 5 (Very Severe):
What was the level of your fatigue on most days?
Sum of those 4 Questions make up the Experience Subdomain Score Ranging from 4 to 20
Social Impact Subdomain Score
   2 Questions with Scores Ranging from 1 (Not at All) to 5 (Very Much):
To what degree did your fatigue interfere with your social activities?
To what degree did your fatigue interfere with your ability to engage in recreational activities?
   2 Questions with Scores Ranging from 1 (Never) to 5 (Always):
How often did you have to limit your social activities because of your fatigue?
How often were you too tired to socialize with your friends?
Sum of those 4 Questions make up the Social Impact Subdomain Score Ranging from 4 to 20
Motivational Impact Subdomain Score
   2 Questions with Scores Ranging from 1 (Not at All) to 5 (Very Much):
To what degree did you have trouble starting things because of your fatigue?
To what degree did you have trouble finishing things because of your fatigue?
   2 Questions with Scores Ranging from 1 (Never) to 5 (Always):
How often were you less effective at home due to your fatigue?
How often did you have to push yourself to get things done because of your fatigue?
Sum of those 4 Questions make up the Motivational Impact Subdomain Score Ranging from 4 to 20
Cognitive Impact Subdomain Score
   2 Questions with Scores Ranging from 1 (Not at All) to 5 (Very Much):
To what degree did your fatigue make it difficult to make decisions?
To what degree did your fatigue make you feel slowed down in your thinking?
   2 Questions with Scores Ranging from 1 (Never) to 5 (Always):
How often were you too tired to think clearly?
How often did your fatigue make you more forgetful?
Sum of those 4 Questions make up the Cognitive Impact Subdomain Score Ranging from 4 to 20

If at least 2 questions were answered within an individual subdomain, that subdomain score was imputed if missing responses are present. Each subdomain score was multiplied by 4/(number of questions answered) and rounded off to the nearest whole number. Otherwise, that subdomain score was set to missing. The total score was calculated by taking the sum of those 4 subdomains and ranged from 16 to 80. If any of the subdomains were missing, the total score was set to missing. The PROMISFM fatigue profile assessment was collected and analyzed at visits Screening, Day 1, Week 2, Week 4, Week 6, and Follow-up (Week 7). The observed total and subdomain scores, changes from baseline, and changes from treatment periods were summarized using descriptive statistics. Additionally, the treatment group comparisons were analyzed using the paired t-test and nonparametric Wilcoxon signed-rank test. Data collected during an Early Termination visit were not summarized in the tables. All PROMISFM fatigue profile data collected and the calculated total and subdomain scores at each visit were presented in a subject listing. With the exception of placebo at Week 2, the exploratory endpoints of interest with this data were as described above.

As used herein, “MISCI” is Multidimensional Inventory of Subject Cognitive Impairment. The MISCI is a 10-item assessment that measures cognitive function. The instrument examines 5 cognitive domains: mental clarity, memory, language, executive functioning, and attention/concentration. The first 6 items are positively worded and reflect perceived cognitive abilities and the last 4 items are negatively worded and reflect perceived cognitive difficulties. The same scoring metric is used for both response scales. However, the scores on items 7-10 must be reverse-coded prior to summing the responses to generate the total score due to the negative wording of these items. Here is a list of the categorical responses with the numeric values that make up the total score:
Positively Worded Items 1-6:“Not at All”=1; “A Little Bit”=2; “Somewhat”=3; “Quite a Bit”=4; “Very Much”=5
Negatively Worded Items 7-10: “Never”=5; “Rarely”=4; “Sometimes”=3; “Often”=2; “Very Often”=1

The total score was calculated by the sum of all the 10 items ranging from 10 to 50, with higher scores indicating better perceived cognitive functioning (i.e. lower impairment). If any of the 10 items were missing, then the total score was set to missing. No imputation methods for missing data were applied for this data. The MISCI assessment was collected and analyzed at visits Screening, Day 1, Week 2, Week 4, Week 6, and Follow-up (Week 7). The observed total score, changes from baseline, and changes from treatment periods were summarized using descriptive statistics. Additionally, the treatment group comparisons were analyzed using the paired ttest and nonparametric Wilcoxon signed-rank test. Data collected during an Early Termination visit were not summarized in the tables. With the exception of placebo at Week 2, the exploratory endpoints of interest with this data were as described above.

All 10 individual item responses and the calculated total score at each visit were presented in a subject listing.

As used herein, “FIQR” is Revised Fibromyalgia Impact Questionnaire. The FIQR is an updated version of the Fibromyalgia Impact Questionnaire which is an assessment of the physical functioning, work status, depression, anxiety, morning tiredness, pain, stiffness, fatigue and wellbeing in fibromyalgia subjects measured over the period of 1 week. The FIQR differs from the FIQ in that it includes questions on memory, tenderness, balance, and environmental sensitivity as well as modified function questions. All 21 FIQR questions are based on an 11-point numerical rating scale/Likert scale ranging from 0 to 10. Lower scores represent a better quality of life. The FIQR is made of the 3 following domains:
Function Domain Score
9 Questions with Scores Ranging from 0 (No Difficulty) to 10 (Very Difficult)
Sum of those 9 Questions make up the Function Domain Score Ranging from 0 to 90
Missing Data Imputation Factor: If at least 5 questions were answered, the Function Domain Score was multiplied by 9/(number of questions answered) and rounded off to the nearest whole number. Otherwise, the score was set to missing.
Overall Impact Domain Score
2 Questions with Scores Ranging from 0 (Never) to 10 (Always)
Sum of those 2 Questions make up the Overall Impact Domain Score Ranging from 0 to 20
Missing Data Imputation Factor: If 1 question was missing, the Overall Impact Domain Score was multiplied by 2. Otherwise, the score was set to missing.
Symptoms Domain Score
10 Questions with Scores Ranging from 0 to 10 with Different Categorical Reponses Sum of those 10 Questions make up the Symptoms Domain Score Ranging from 0 to 100 Missing Data Imputation Factor: If at least 5 questions were answered, the Function Domain Score was multiplied by 10/(number of questions answered) and rounded off to the nearest whole number. Otherwise, the score was set to missing.

If all 3 domain scores could be calculated, the Total FIQR Score ranged from 0 to 100 and was calculated based on the normalization domain scores: (Function Domain Score/3)+Overall Impact Domain Score+(Symptoms Domain Score/2). Otherwise, the Total FIQR Score was set to missing. The Function and Symptoms domain scores collected on the CRF applied the normalization factors stated above (Function Domain Score/3 and Symptoms Domain Score/2) in order to calculate the Total FIQR Score on the CRF. As a result, the Function and Symptoms domain scores were derived by the definition stated above in the SAP as opposed to using the collected values on the CRF. The FIQR assessment was collected and analyzed at visits Screening, Day 1, Week 2, Week 4, Week 6, and Follow-up (Week 7). The observed total and domain scores, changes from baseline, and changes from treatment periods were summarized using descriptive statistics. Additionally, the treatment group comparisons were analyzed using the paired ttest and nonparametric Wilcoxon signed-rank test. Data collected during an Early Termination visit were not summarized in the tables. All FIQR data collected and the calculated total and domain scores at each visit were presented in a subject listing. With the exception of placebo at Week 2, the exploratory endpoints of interest with this data were as described above.

As used herein, the “PROMIS Sleep Disturbance” short form is Patient Reported Outcomes Measurement Information System Sleep Disturbance—Short Form. The PROMIS Sleep Disturbance short form version 1.0 8b is an 8-item instrument that assesses sleep disturbance in subjects 18 years and older. All 8 questions are based on a 5-point numerical rating scale/Likert scale ranging from 1 to 5 with different categorical responses. Lower scores represent a better quality of life. The total score was calculated by taking the sum of all 8 questions and ranged from 8 to 40. If at least 4 questions were answered, the total score was imputed if missing responses were present. The total score was multiplied by 8/(number of questions answered) and rounded off to the nearest whole number. Otherwise, the total score was set to missing.

The PROMIS Sleep Disturbance short form assessment was collected and analyzed at visits Screening, Day 1, Week 2, Week 4, Week 6, and Follow-up (Week 7). The observed total score, changes from baseline, and changes from treatment periods were summarized using descriptive statistics. Additionally, the treatment group comparisons were analyzed using the paired t-test and nonparametric Wilcoxon signed-rank test. Data collected during an Early Termination visit were not summarized in the tables. All PROMIS Sleep Disturbance short form data collected and the calculated total score at each visit were presented in a subject listing. With the exception of placebo at Week 2, the exploratory endpoints of interest with this data were as described above.

As used herein, “HADS” is Hospital Anxiety and Depression Scale. The HADS is a self-reported screening tool for anxiety and depression in non-psychiatric clinical populations. The scale consists of 14 items (7 each for anxiety and depression), each rated on a 4-point scale ranging from 0 to 3 (i.e. 0=not at all; 3=most of the time). The categorical responses are slightly different for some of the questions, but the numeric scores remained the same from 0 to 3. Responses were based on the relative frequency of symptoms over the preceding week. When a HADS questionnaire was partially incomplete, the individual subscale scores were estimated by assuming that the missing item(s) had a value equal to the average of those in existence, provided that no more than 3 items of a subscale were missing. The individual imputed missing scores used the specific subscale mean score and were rounded off to the nearest whole number. If an individual subscale score was missing 4 or more items, then that subscale score was set to missing. A total score adding the anxiety and depression subscale scores for HADS was not calculated. Possible scores range from 0 to 21 for each subscale. An analysis of scores on the 2 subscales supported the differentiation of each mood state into 4 ranges:
Normal Cases (Scores 0-7)
Mild Cases (Scores 8-10)
Moderate Cases (Scores 11-14)
Severe Cases (Scores 15-21)

The HADS assessment was collected and analyzed at visits Screening, Day 1, Week 2, Week 4, Week 6, and Follow-up (Week 7). The observed anxiety and depression subscale scores, changes from baseline, and changes from treatment periods were summarized using descriptive statistics. Additionally, the treatment group comparisons were analyzed using the paired t-test and nonparametric Wilcoxon signed-rank test. Data collected during an Early Termination visit were not summarized in the tables. With the exception of placebo at Week 2, the exploratory endpoints of interest with this data were as described above.

The 4 interpreted categorical values for each subscale were summarized in a frequency distribution table. All HADS data collected and the calculated subscale scores at each visit were presented in a subject listing.

As used herein, the “PROMIS Physical Function short form” is Patient Reported Outcomes Measurement Information System Physical Function Short Form. The PROMIS physical function short form version 1.0 12a measures the self-reported performance of physical activities including dexterity, walking or mobility and the ability to complete activities of daily living. All 12 questions are based on a 5-point numerical rating scale/Likert scale ranging from 1 to 5 with different categorical responses. Higher scores represent a better quality of life. The PROMIS physical function is made of the 2 following subdomains:
Walking Subdomain Score
If the question, “Can you walk 25 feet on a level surface (with or without support)?” is answered yes, then 6 questions will be assessed. Otherwise, the Walking Subdomain Score will not be calculated.
   4 Questions with Scores Ranging from 1 (Unable to do) to 5 (Without any difficulty):
Are you able to walk on flat ground?
Are you able to walk up and down two steps?
Are you able to run at a fast pace for 2 miles?
Are you able to do yard work like raking leaves, weeding, or pushing a lawn mower?
   2 Questions with Scores Ranging from 1 (Cannot do) to 5 (Not at all):
Does your health now limit you in doing strenuous activities such as backpacking, skiing, playing tennis, bicycling, or jogging?
Does your health now limit you in hiking a couple of miles on uneven surfaces, including hills?
Sum of those 6 Questions make up the Walking Subdomain Score Ranging from 6 to 30
Other Physical Function Subdomain Score
   5 Questions with Scores Ranging from 1 (Unable to do) to 5 (Without any difficulty):
Are you able to wash and dry your body?
Are you able to get in and out of bed?
Are you able to bend down and pick up clothing from the floor?
Are you able to push open a heavy door?
Are you able to reach and get down an object (such as a can of soup) from above your head?
   1 Question with Scores Ranging from 1 (Cannot do) to 5 (Not at all):
Does your health now limit you in doing eight hours of physical labor?
Sum of those 6 Questions make up the Other Physical Function Subdomain Score Ranging from 6 to 30

If at least 3 questions have been answered within an individual subdomain, that subdomain score may be imputed if missing responses are present. Each subdomain score was multiplied by 6/(number of questions answered) and rounded off to the nearest whole number. Otherwise, that subdomain score was set to missing. A total score for PROMIS physical function was not calculated. The PROMIS physical function assessment was collected and analyzed at visits Screening, Day 1, Week 2, Week 4, Week 6, and Follow-up (Week 7). The observed subdomain scores, changes from baseline, and changes from treatment periods were summarized using descriptive statistics. Additionally, the treatment group comparisons were analyzed using the paired t-test and nonparametric Wilcoxon signed-rank test. Data collected during an Early Termination visit were not summarized in the tables. All PROMIS physical function data collected and the calculated subdomain scores at each visit were presented in a subject listing. With the exception of placebo at Week 2, the exploratory endpoints of interest with this data were as described above.

As used herein, the “PGI-C” is Patient Global Impression of Change. The PGI-C is a 7-point Likert scale that allows subjects to rate the change in the disease state from study initiation to specific time points during the study or at the end of the study. It provides the subject's impression of overall change. Below are the numeric values assigned in the 7-point scale along with categorical values and analysis categorical values:
1 Very Much Improved
2 Much Improved
3 Minimally Improved Minimally Improved to Worse
4 No Change Minimally Improved to Worse
5 Minimally Worse Minimally Improved to Worse
6 Much Worse Minimally Improved to Worse
7 Very Much Worse Minimally Improved to Worse
Missing Not Reported

The PGI-C data were collected and analyzed at visits Week 2, Week 4, Week 6, and Follow-up (Week 7). There is no baseline collection due to the nature of this data being the change after receiving study drug. The actual values and changes from treatment periods at Week 4 and Week 6 were summarized using descriptive statistics. A frequency distribution was created for each analysis visit based on the collected categorical values. The analysis categorical values stated above were summarized by frequency distributions and p-values using McNemar's test. Data collected during an Early Termination visit were not summarized in the tables. The exploratory endpoints of interest with this data is the change in scores at the following analysis visits:
NYX-2925 200 mg PO QD at Week 6 Change from Week 4 (NYX-2925 20 mg PO QD)
Follow-Up at Week 7 Change from Week 6 (NYX-2925 200 mg PO QD)

As used herein, “FSQ” is Fibromyalgia Survey Questionnaire, which is a patient-reported tool to assess the 2011 ACR Fibromyalgia criteria circumventing the requirement of physician assessment. The criteria review the widespread pain index, and somatic symptoms. The somatic symptoms of fatigue, waking unrefreshed, and cognitive symptoms were scored from 0 (no problem) to 3 (severe, pervasive, continuous, life disturbing problems). Symptoms of headaches, pain or cramps in the lower abdomen, and depression were given a score of 1, if they had occurred in the past 3 months.

As used herein, “WPI” is the Widespread Pain Index, which is a score comprised of 19 areas where subjects experienced pain within the last week with a yes or no response for each area. Each individual yes response has a numeric value of 1 and 0 is assigned to each no response. The WPI score had values ranging from 0 to 19. If any of the individual area responses were missing, the WPI score was not calculated. Here are the 19 areas that were assessed: Left Jaw, Right Jaw, Left Shoulder, Right Shoulder, Right Upper Arm, Left Upper Arm, Right Lower Arm, Left Lower Arm, Neck, Upper Back, Chest, Abdomen, Lower Back, Right Hip, Left Hip, Right Upper Leg, Left Upper Leg, Right Lower Leg, and Left Lower Leg.

As used herein, “SS” is the Symptom Severity Score, based on 3 questions each having a numeric score ranging from 0 to 3 and an additional 3 questions each having a numeric score of 0 or 1. If all 6 questions were given a numeric score, then the Symptom Severity Score ranged from 0 to 12. If any of the individual scores were missing, the Symptom Severity Score was not calculated. No imputation methods for missing data were applied for this data.

As used herein, “ACR Fibromyalgia Criteria” is 2011 American College of Rheumatology Fibromyalgia Criteria, and includes FSQ, WPI, and SS. These assessments were analyzed at Day 1, Week 2, Week 4, Week 6, and Follow-up (Week 7). Both the observed Widespread Pain Index score and Symptom Severity Score, changes from baseline, and changes from treatment periods were summarized using descriptive statistics. Additionally, the treatment group comparisons were analyzed using the paired t-test and nonparametric Wilcoxon signed-rank test. A frequency distribution table was presented for yes/no responses for these categories:
Widespread Pain Index ≥7 and Symptom Severity Score >5
Widespread Pain Index is 3 to 6 and the Symptom Severity Score is >9

Data collected during an Early Termination visit were not summarized in the tables. All the data collected for the 2011 American College of Rheumatology Fibromyalgia Criteria were provided in an individual subject listing. With the exception of placebo at Week 2, the exploratory endpoints of interest with this data were as described above.

### Pharmaceutical Formulations

In one aspect, provided herein is a pharmaceutical formulation comprising (2S, 3R)-3-hydroxy-2-((R)-5-isobutyryl-1-oxo-2,5-diazaspiro[3.4]octan-2-yl)butanamide (“NYX-2925”), or a pharmaceutically acceptable salt thereof, and a pharmaceutically acceptable excipient, which can be used for the treatment of fibromyalgia.

In some embodiments, the pharmaceutical formulation comprises NYX-2925, or a pharmaceutically acceptable salt thereof, present in a therapeutically effective amount; and a pharmaceutically acceptable excipient. In some embodiments, the pharmaceutically acceptable excipient is one or more of microcrystalline cellulose; pregelatinized starch, and magnesium stearate.

In some embodiments, the pharmaceutical formulation is encapsulated in a capsule. In some embodiments, the capsule comprises hydroxyl-propyl cellulose. In some embodiments, the capsule comprises about 20 mg of NYX-2925, or a pharmaceutically acceptable salt thereof; about 50 mg of NYX-2925, or a pharmaceutically acceptable salt thereof; about 100 mg NYX-2925, or a pharmaceutically acceptable salt thereof; or about 200 mg NYX-2925, or a pharmaceutically acceptable salt thereof.

In some embodiments, the pharmaceutical formulation is in the form of a tablet. In some embodiments, the tablet comprises about 20 mg of NYX-2925, or a pharmaceutically acceptable salt thereof; about 50 mg of NYX-2925, or a pharmaceutically acceptable salt thereof; about 100 mg NYX-2925, or a pharmaceutically acceptable salt thereof; or about 200 mg NYX-2925, or a pharmaceutically acceptable salt thereof.

In certain embodiments, the amount of NYX-2925, or a pharmaceutically acceptable salt thereof, in the pharmaceutical formulation is from about 20 mg to about 150 mg, about 50 mg to about 150 mg, about 75 mg to about 150 mg, about 100 mg to about 150 mg, about 125 mg to about 150 mg, about 25 mg to about 125 mg, about 25 mg to about 100 mg, about 25 mg to about 75 mg, about 25 mg to about 50 mg, about 50 mg to about 125 mg, about 50 mg to about 100 mg, about 50 mg to about 75 mg, about 75 mg to about 125 mg, about 75 mg to about 100 mg, or about 100 mg to about 125 mg.

In certain embodiments, the amount of NYX-2925, or a pharmaceutically acceptable salt thereof, in the pharmaceutical formulation is about 20 mg, about 50 mg, about 75 mg, about 100 mg, about 125 mg, about 150 mg, about 175 mg, or about 200 mg. In some embodiments, the amount of NYX-2925, or a pharmaceutically acceptable salt thereof, in the pharmaceutical formulation is about 50 mg. In some embodiments, the amount of NYX-2925, or a pharmaceutically acceptable salt thereof, in the pharmaceutical formulation is about 200 mg. In some embodiments, the amount of NYX-2925, or a pharmaceutically acceptable salt thereof, in the pharmaceutical formulation is about 100 mg. In some embodiments, the amount of NYX-2925, or a pharmaceutically acceptable salt thereof, in the pharmaceutical formulation is about 50 mg. In some embodiments, the amount of NYX-2925, or a pharmaceutically acceptable salt thereof, in the pharmaceutical formulation is about 20 mg.

In certain embodiments, the pharmaceutical formulation comprises an effective amount of a pharmaceutically acceptable salt of NYX-2925.

In certain embodiments, the pharmaceutical formulation comprises an effective amount of a pharmaceutically acceptable solvate of NYX-2925, or a solvate of a pharmaceutically acceptable salt of NYX-2925. In some embodiments, the solvate is a hydrate. In some embodiments, the solvate is a monohydrate.

In certain embodiments, the pharmaceutical formulation provided herein can be in a solid dosage form. In some embodiments, the solid dosage form is a capsule.

In certain embodiments, provided herein is a capsule comprising a pharmaceutical formulation, the pharmaceutical formulation comprising about 10 mg of NYX-2925, or a pharmaceutically acceptable salt thereof; about 50 mg of NYX-2925, or a pharmaceutically acceptable salt thereof; about 100 mg NYX-2925, or a pharmaceutically acceptable salt thereof; or about 200 mg NYX-2925, or a pharmaceutically acceptable salt thereof; and a pharmaceutically acceptable excipient.

In some embodiments, about 20 mg of NYX-2925 or a pharmaceutically acceptable salt thereof is provided as a unit dose. In some embodiments, about 50 mg of NYX-2925 or a pharmaceutically acceptable salt thereof is provided as a unit dose. In some embodiments, about 100 mg of NYX-2925 or a pharmaceutically acceptable salt thereof is provided as a unit dose. In some embodiments, about 200 mg of NYX-2925 or a pharmaceutically acceptable salt thereof is provided as a unit dose.

In certain embodiments, the pharmaceutical formulation comprises NYX-2925 or a pharmaceutically acceptable salt thereof; and a pharmaceutically acceptable excipient.

In certain embodiments, the pharmaceutical formulation is a formulation for oral administration.

Although the descriptions of pharmaceutical formulations provided herein are principally directed to pharmaceutical formulations which are suitable for administration to humans, it will be understood by the skilled artisan that such formulations are generally suitable for administration to animals of all sorts. Modification of pharmaceutical formulations suitable for administration to humans in order to render the formulations suitable for administration to various animals is well understood, and the ordinarily skilled veterinary pharmacologist can design and/or perform such modification with ordinary experimentation. General considerations in the formulation and/or manufacture of pharmaceutical formulations can be found, for example, in Remington: The Science and Practice of Pharmacy 21^{st} ed., Lippincott Williams & Wilkins, 2005.

### Methods of Use and Treatment

In one aspect, provided herein are methods of treating fibromyalgia in a patient in need thereof, comprising administering daily to the patient an amount of (2S, 3R)-3-hydroxy-2-((R)-5-isobutyryl-1-oxo-2,5-diazaspiro[3.4]octan-2-yl)butanamide (“NYX-2925”), or a pharmaceutically acceptable salt thereof, wherein the amount is between about 20 mg and about 200 mg.

In some embodiments, the amount of NYX-2925 is between about 25 mg to about 180 mg, between about 25 mg to about 160 mg, between about 30 mg to about 140 mg, between about 35 mg to about 120 mg, between about 40 mg to about 100 mg, between about 45 mg to about 80 mg, or between about 50 mg to about 60 mg. In some embodiments, the amount is between about 44 mg to about 56 mg, between about 45 mg to about 55 mg, between about 46 mg to about 54 mg, between about 47 mg to about 53 mg, between about 48 mg to about 52 mg, or between about 49 mg to about 51 mg. In some embodiments, the amount is about 20 mg, about 50 mg, about 100 mg, or about 200 mg.

In some embodiments after daily administration of NYX-2925, or a pharmaceutically acceptable salt thereof, the patient has a reduction in levels of resting Glx in the dorsal anterior cingulate cortex after an evoking pain stimulus, wherein the level of Glx is the sum of a glutamate level and a glutamine level in the patient.

In some embodiments, after daily administration of NYX-2925, or a pharmaceutically acceptable salt thereof, the patient has a reduction in the increase of levels of Glx in the posterior insular cortex after an evoking pain stimulus, wherein the level of Glx is the sum of a glutamate level and a glutamine level in the patient.

In some embodiments, the patient has reduced levels of Glx in key pain-regulating brain regions after daily administration of NYX-2925, or a pharmaceutically acceptable salt thereof, wherein the level of Glx is the sum of a glutamate level and a glutamine level in the patient.

In some embodiments, after daily administration of NYX-2925, or a pharmaceutically acceptable salt thereof, the patient has a reduced average daily pain score. In some embodiments, after daily administration of NYX-2925, or a pharmaceutically acceptable salt thereof, for four weeks, the patient has a statistically-significant reduced average daily pain score.

In some embodiments, after daily administration of NYX-2925, or a pharmaceutically acceptable salt thereof, the patient has a reduced worst daily pain score. In some embodiments, after daily administration of NYX-2925, or a pharmaceutically acceptable salt thereof, for four weeks, the patient has a statistically-significant reduced worst daily pain score.

In some embodiments, after daily administration of NYX-2925, or a pharmaceutically acceptable salt thereof, the patient has a reduced Revised Fibromyalgia Impact Questionnaire (“FIQR”) score. In some embodiments, after daily administration of NYX-2925, or a pharmaceutically acceptable salt thereof, for four weeks, the patient has a statistically-significant reduced FIQR score.

In some embodiments, after daily administration of NYX-2925, or a pharmaceutically acceptable salt thereof, the patient has a reduced PROMIS_{FM} Fatigue Profile total score. In some embodiments, after daily administration of NYX-2925, or a pharmaceutically acceptable salt thereof, for four weeks, the patient has a statistically-significant reduced PROMIS_{FM} Fatigue Profile total score.

In some embodiments, the patient has reduced sleep interference after about 1 week, after about 2 weeks, after about 3 weeks, after about 4 weeks, or after about 5 weeks or more of daily administration of NYX-2925, or a pharmaceutically acceptable salt thereof.

In some embodiments, the patient is not being administered another analgesic. In some embodiments, the patient is being administered another analgesic.

In some embodiments, administering comprises administering daily to the patient a pharmaceutical formulation, wherein the pharmaceutical formulation comprises NYX-2925, or a pharmaceutically acceptable salt thereof, present in a therapeutically effective amount; microcrystalline cellulose; pregelatinized starch, and magnesium stearate.

In some embodiments, the pharmaceutical formulation is encapsulated in a capsule. In some embodiments, the capsule comprises hydroxyl-propyl cellulose. In some embodiments, the capsule comprises about 10 mg of NYX-2925, or a pharmaceutically acceptable salt thereof; about 20 mg of NYX-2925, or a pharmaceutically acceptable salt thereof; about 50 mg of NYX-2925, or a pharmaceutically acceptable salt thereof; about 100 mg NYX-2925, or a pharmaceutically acceptable salt thereof; or about 200 mg NYX-2925, or a pharmaceutically acceptable salt thereof.

In some embodiments, the pharmaceutical formulation is in the form of a tablet. In some embodiments, the tablet comprises about 10 mg of NYX-2925, or a pharmaceutically acceptable salt thereof; about 20 mg of NYX-2925, or a pharmaceutically acceptable salt thereof; about 50 mg of NYX-2925, or a pharmaceutically acceptable salt thereof; about 100 mg NYX-2925, or a pharmaceutically acceptable salt thereof; or about 200 mg NYX-2925, or a pharmaceutically acceptable salt thereof.

In some embodiments, the patient is human.

The pharmaceutical methods provided herein can include administration by oral (enteral) administration. That is, in some embodiments, the methods include administering orally a compound or a pharmaceutical formulation disclosed herein.

The methods provided herein may also include chronic administration. Chronic administration refers to administration of a compound or pharmaceutical formulation comprising a compound disclosed herein over an extended period of time, e.g., for example, over 3 months, 6 months, 1 year, 2 years, 3 years, 5 years, etc., or may be continued indefinitely, for example, for the rest of the subject's life. In certain embodiments, the chronic administration is intended to provide a constant level of the compound in the blood, e.g., within the therapeutic window over the extended period of time.

The pharmaceutical formulations provided herein may be presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions.

In certain embodiments, the pharmaceutical formulations provided herein are administered to the patient as a solid dosage form. In some embodiments, the solid dosage form is a capsule.

In certain embodiments, the compounds provided herein can be administered as the sole therapeutically active agent, or they can be administered in combination with other therapeutically active agents.

In certain embodiments, administering an effective amount of NYX-2925, or a pharmaceutically acceptable salt thereof, comprises administering orally about 20 mg of NYX-2925, or a pharmaceutically acceptable salt thereof, once daily.

In certain embodiments, administering an effective amount of NYX-2925, or a pharmaceutically acceptable salt thereof, comprises administering orally about 50 mg of NYX-2925, or a pharmaceutically acceptable salt thereof, once daily.

In certain embodiments, administering an effective amount of NYX-2925, or a pharmaceutically acceptable salt thereof, comprises administering orally about 100 mg of NYX-2925, or a pharmaceutically acceptable salt thereof, once daily.

In certain embodiments, administering an effective amount of NYX-2925, or a pharmaceutically acceptable salt thereof, comprises administering orally about 200 mg of NYX-2925, or a pharmaceutically acceptable salt thereof, once daily.

In some embodiments, the about 20 mg of NYX-2925 or a pharmaceutically acceptable salt thereof is provided as a unit dose. In some embodiments, the about 20 mg of NYX-2925 or a pharmaceutically acceptable salt thereof is provided as a 10 mg unit dose and a 10 mg unit dose.

In some embodiments, the about 100 mg of NYX-2925 or a pharmaceutically acceptable salt thereof is provided as a unit dose. In some embodiments, the about 100 mg of NYX-2925 or a pharmaceutically acceptable salt thereof is provided as a 50 mg unit dose and a 50 mg unit dose.

In some embodiments, the about 200 mg of NYX-2925 or a pharmaceutically acceptable salt thereof is provided as a unit dose. In some embodiments, the about 200 mg of NYX-2925 or a pharmaceutically acceptable salt thereof is provided as a 100 mg unit dose and a 100 mg unit dose.

Also provided herein are combination therapies comprising a compound of the disclosure, e.g., NYX-2925, in combination with one or more other active agents. For example, a compound may be combined with one or more analgesics, such as non-steroidal anti-inflammatory agents (NSAIDS), steroidal anti-inflammatory agents, opiates, and cyclo-oxygenase inhibitors. A compound may also be combined with other agents such as antidepressants, such as tricyclic antidepressants, MAO-I's, SSRI's, SNRI's, and double and triple uptake inhibitors and/or anxiolytic drugs. Exemplary drugs that may be used in combination with a compound include Anafranil, Adapin, Aventyl, Elavil, Norpramin, Pamelor, Pertofrane, Sinequan, Surmontil, Tofranil, Vivactil, Parnate, Nardil, Marplan, Celexa, Lexapro, Luvox, Paxil, Prozac, Zoloft, Wellbutrin, Effexor, Remeron, Cymbalta, Desyrel (trazodone), and Ludiomill. In another example, a compound may be combined with an antipsychotic medication. Non-limiting examples of antipsychotics include butyrophenones, phenothiazines, thioxanthenes, clozapine, olanzapine, risperidone, quetiapine, ziprasidone, amisulpride, asenapine, paliperidone, iloperidone, zotepine, sertindole, lurasidone, and aripiprazole. In another example, a compound, e.g., NYX-2925, may be combined with an antiepileptic medication. Non-limiting examples of antiepileptics include gabapentin, pregabalin, carbamazepine, clonazepam, lacosamide, lamotrigine, levetiracetam, oxcarbazepine, phenytoin, pregabalin, topiramate, and valproate. Use of any of the drugs named herein can include use of its branded or generic equivalents. It should be understood that combinations of a compound, e.g., NYX-2925, and one or more of the above therapeutics may be used for treatment of any suitable condition and are not limited to use as anti-pain medication.

### EXAMPLES

In order that the disclosure described herein may be more fully understood, the following examples are set forth. The synthetic and biological examples described in this application are offered to illustrate the compounds, pharmaceutical formulations, and methods provided herein and are not to be construed in any way as limiting their scope.

The compounds described herein can be prepared in a number of ways based on the teachings contained herein and synthetic procedures known in the art. The following non-limiting examples illustrate the disclosure.

All sample preparation and measurement was done in ambient air atmosphere at about one atmosphere of pressure.

### Chemical Definitions

Herein, the following chemical definitions are used: “Bn” is benzyl, “Boc” is tert-butoxycarbonyl, “BOM” is benzyloxymethyl, “DCM” is dichloromethane, “DIAD” is diisopropyl azodicarboxylate, “DIPEA” is N,N-diisopropylethylamine, “EDCI” is 1-ethyl-3-(3-dimethylaminopropyl)carbodiimide, “HATU” is (1-[Bis(dimethylamino)methylene]-1H-1,2,3-triazolo[4,5-b]pyridinium 3-oxid hexafluorophosphate, “¹H-NMR” is proton nuclear magnetic resonance, “HUM” is hydroxybenzotriazole, “LCMS” is liquid chromatography-mass spectrometry, “LiHMDS” is lithium bis(trimethylsilyl)amide, “RT” is room temperature, “TEA” is triethylamine, “TFA” is trifluoroacetic acid, and “TLC” is thin-layer chromatography.

### Example 1: Synthesis of NYX-2925

### Scheme 1—Synthesis of Intermediate 2S-L

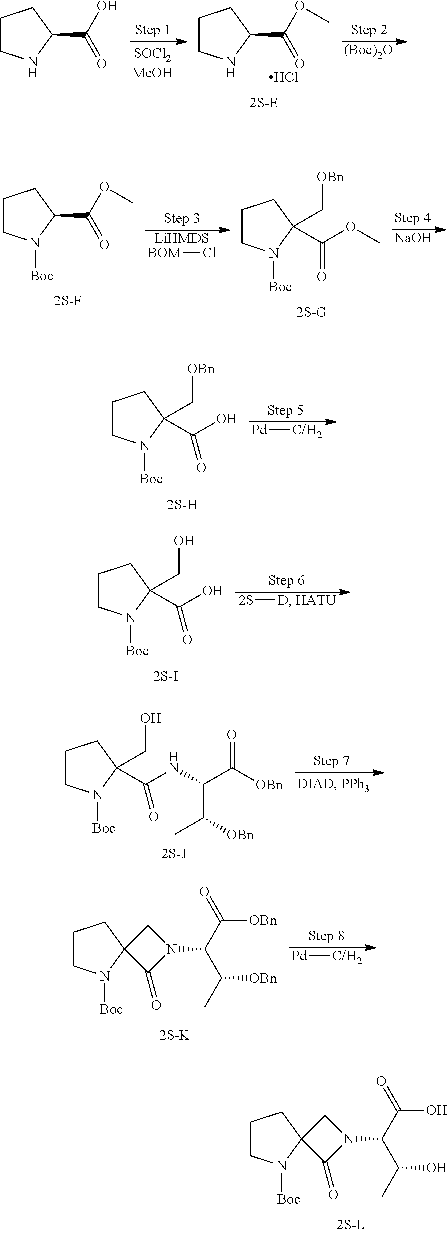

### Synthesis of methyl pyrrolidine-2-carboxylate (2S-E)

To a stirring solution of L-proline (50 g, 434 mmol) in methanol was added thionyl chloride (37.5 ml, 521 mmol) at 0° C. and heated to 70° C. for 16 h. The reaction mixture was brought to RT and concentrated under vacuum to afford compound 2S-E as (70 g, 99%) as thick syrup (hydrochloride salt).

¹H-NMR: (500 MHz, DMSO-d₆): δ 4.15-4.13 (m, 1H), 3.65 (s, 3H), 3.35-3.30 (m, 2H), 2.23-2.15 (m, 1H), 1.86-1.78 (m, 3H), 1.41 (s, 9H);

LCMS m/z: 129 [M⁺+1]

### Synthesis of 1-tert-butyl 2-methyl pyrrolidine-1, 2-dicarboxylate (2S-F)

To a stirring solution of compound 2S-E (70 g, 422 mmol) in CH₂Cl₂ (700 mL) were added Et₃N (183 mL, 1.26 mol) at 0° C. and stirred for 10 min. After added Boc-anhydride (184 mL, 845 mmol) at 0° C. and the reaction mixture was stirred at RT for 16 h. After consumption of the starting material (by TLC), the reaction was diluted with water (200 mL) and extracted with CH₂Cl₂ (2×200 mL). The combined organic layer was washed with citric acid (1×150 mL), brine (1×200 mL). The organic layer was dried over Na₂SO₄ and concentrated under reduced pressure to afford crude compound which was purified by column chromatography by eluting 50% EtOAc/n-hexane to obtain compound 2S-F (80 g, 83%) as thick syrup.

¹H-NMR: (400 MHz, DMSO-d₆): δ 4.15-4.13 (m, 1H), 3.65 (s, 3H), 3.35-3.30 (m, 2H), 2.23-2.15 (m, 1H), 1.86-1.78 (m, 3H), 1.41 (s, 9H);

LCMS m/z: 229 [(M⁺+1)-Boc].

### Synthesis of 1-tert-butyl 2-methyl 2-((benzyloxy) methyl) pyrrolidine-1, 2-dicarboxylate (2S-G)

To a stirring solution of compound 2S-F (25 g, 109 mmol) in THF (250 mL) was added LiHMDS (240 mL, 240 mmol) at −20° C. and stirred for 2 h. To this BOM-chloride (23 mL, 163 mmol) was added drop wise at −30° C. and stirred for 2 h. After consumption of the starting material (by TLC), the reaction was quenched with aqueous NH₄Cl solution (100 mL) and extracted with EtOAc (2×200 mL). The combined organic layer was washed with water (2×150 mL) followed by brine solution (2×100 mL). The organic layer was dried over Na₂SO₄ and concentrated to obtain crude compound which was purified by column chromatography by eluting 10% EtOAc/n-hexane to afford compound 25-G (30 g, 79%) as thick syrup.

¹H-NMR: (500 MHz, DMSO-d₆): δ 7.36-7.22 (m, 5H), 4.59-4.48 (m, 2H), 4.02-3.88 (m, 1H), 3.63 (s, 3H), 3.49-3.35 (m, 2H), 3.34-3.30 (m, 1H), 2.31-2.23 (m, 1H), 2.04-1.89 (m, 2H), 1.82-1.78 (m, 1H);

LCMS m/z: 349.4 [(M⁺+1)-Boc]

### Synthesis of 2-((benzyloxy) methyl)-1-(tert-butoxycarbonyl) pyrrolidine-2-carboxylic acid (2S-H)

To a stirring solution of compound 25-G (30 g, 86 mmol) in methanol (70 mL) was added NaOH solution (6.88 g in 70 mL H₂O) at RT. The reaction mixture was heated to 70° C. for 16 h. After consumption of the starting material (by TLC), the solvent from the reaction was evaporated under reduced pressure and diluted with EtOAc (2×200 mL). The separated aqueous layer was acidified using citric acid solution (pH-3) and extracted with EtOAc (2×250 mL).

The combined organic layer was dried over Na₂SO₄ and concentrated to afford crude was triturated with n-hexane to obtain compound 2S-H (25 g, 86.8%) as an off-white solid.

¹H-NMR: (400 MHz, DMSO-d₆): δ 12.35 (br s, 1H), 7.37-7.29 (m, 5H), 4.56-4.48 (m, 2H), 4.06-4.00 (m, 1H), 3.92-3.89 (m, 1H), 3.66-3.45 (m, 1H), 3.37-3.28 (m, 1H), 2.31-2.20 (m, 1H), 2.05-1.97 (m, 1H), 1.87-1.75 (m, 2H), 1.38 (s, 9H);

LCMS m/z: 335.3 [M⁺+1]

### Synthesis of 1-(tert-butoxycarbonyl)-2-(hydroxymethyl) pyrrolidine-2-carboxylic acid (2S-I)

To a stirring solution of compound 2S-H (25 g, 74 mmol) in methanol (150 mL) was added 50% wet 10% Pd/C (7 g) at RT and stirred for 10 h under H₂ atmosphere. After consumption of the starting material (by TLC), the reaction mixture was filtered through a pad of celite and the pad was washed with methanol (100 mL). Obtained filtrate was concentrated under reduced pressure to afford compound 2S-I (15 g, 82.8%) as white solid.

¹H-NMR: (400 MHz, DMSO-d₆): δ 4.66 (br s, 1H), 3.96-3.83 (m, 1H), 3.63-3.59 (m, 1H), 3.49-3.41 (m, 1H), 3.34-3.25 (m, 1H), 2.30-2.17 (m, 1H), 1.95-1.72 (m, 3H), 1.38 (s, 9H).

Mass (ESI): m/z 245 [M⁺+1]

### Synthesis of tert-butyl 2-(((2S, 3R)-1, 3-bis (benzyloxy)-1-oxobutan-2-yl) carbamoyl)-2-(hydroxymethyl) pyrrolidine-1-carboxylate (2S-J)

To a stirring solution of compound 25-I (18 g, 73.4 mmol) in CH₂Cl₂ (180 mL) were added DIPEA (40 mL, 220 mmol), 2S-D (21.9 g, 73.4 mmol), HATU (41.8 g, 110 mmol) at RT and stirred for 16 h. After consumption of the starting material (by TLC), the reaction mixture was diluted with water (50 mL) and extracted with CH₂Cl₂ (2×100 mL). The combined organic layer was washed with brine, dried over anhydrous Na₂SO₄, filtered and concentrated under reduced pressure. Obtained crude material was purified by silica gel column chromatography eluting with 30% EtOAc/n-hexane to afford compound 2S-J (20 g, 52%) as pale yellow thick syrup.

¹H-NMR: (400 MHz, DMSO-d₆): δ 8.25-8.12 (m, 1H), 7.31-7.27 (m, 10H), 5.85 (t, J=4.8 Hz, 1H), 5.14 (s, 2H), 4.54-4.49 (m, 2H), 4.31-4.20 (m, 1H), 4.15-4.07 (m, 1H), 3.91-3.50 (m, 1H), 3.52-3.37 (m, 1H), 3.31-3.27 (m, 2H), 2.35-2.07 (m, 1H), 1.95-1.90 (m, 1H), 1.73-1.52 (m, 2H), 1.39 (s, 9H), 1.19 (d, J=6.4 Hz, 3H);

Mass (ESI): m/z 527.4 [M⁺+1]

### Synthesis of tert-butyl 2-((2S, 3R)-1, 3-bis (benzyloxy)-1-oxobutan-2-yl)-1-oxo-2, 5-diazaspiro [3.4] octane-5-carboxylate (2S-K)

To a stirring solution of triphenylphosphine (24.7 g, 94 mmol) in THF (100 mL) was added DIAD (15.3 g, 75 mmol) at RT and stirred for 30 min. To this added compound 2S-J (20 g, 37.9 mmol) in (10 mL) THF slowly and reaction mixture was stirred at RT for 2 h. After consumption of the starting material (by TLC), the reaction was concentrated under reduced pressure. The crude material was purified by silica gel column chromatography eluting 25% EtOAc/n-hexane to afford compound 2S-K (17 g, 88%) as pale yellow thick syrup.

¹H-NMR: (400 MHz, DMSO-d₆): δ 7.33-7.26 (m, 5H), 7.23-7.18 (m, 5H), 5.10 (s, 2H), 4.80-4.73 (m, 2H), 4.60 (s, 2H), 4.31 (s, 2H), 4.05-4.00 (m, 2H), 1.80-1.68 (m, 4H), 1.39 (s, 9H), 1.18 (d, J=6.0 Hz, 3H);

Mass (ESI): m/z 509.4 [M⁺+1]

### Synthesis of (2S, 3R)-2-(5-(tert-butoxycarbonyl)-1-oxo-2, 5-diazaspiro [3.4] octan-2-yl)-3-hydroxybutanoic acid (2S-L)

To a stirring solution of compound 2S-K (7 g, 13.7 mmol) in methanol (100 mL) was added 10% Pd/C (4 g) at RT and stirred for 6 h under H₂ atmosphere. After consumption of the starting material (by TLC), the reaction mixture was filtered through a pad of celite and the pad was washed with methanol (50 mL). Obtained filtrate was concentrated under reduced pressure to obtained crude, which was triturated with n-pentane (50 mL) to afford compound 2S-L (4 g, 88%) as white solid.

¹H-NMR: (500 MHz, DMSO-d₆): δ 12.80 (br s, 1H), 4.78-4.73 (m, 1H), 4.21-4.19 (m, 1H), 4.09 (s, 2H), 3.55-3.46 (m, 2H), 2.09-2.05 (m, 2H), 1.80 (d, J=7.0 Hz, 1H), 1.38 (s, 9H), 1.35-1.28 (m, 2H), 1.17 (d, J=6.5 Hz, 3H)

LCMS m/z: 329.6 [M⁺+1]

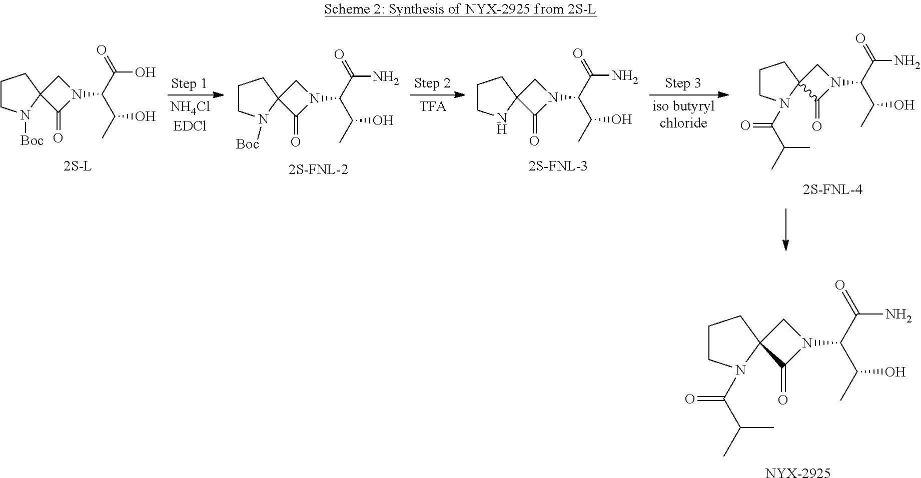

### Synthesis of tert-butyl 2-((2S, 3R)-1-amino-3-hydroxy-1-oxobutan-2-yl)-1-oxo-2, 5-diazaspiro [3.4] octane-5-carboxylate (2S-FNL-2)

To a stirring solution of compound 2S-L (500 mg, 1.52 mmol) in CH₂Cl₂ (5 mL) were added DIPEA (0.8 mL, 4.57 mmol), EDCI.HCl (350 mg, 1.82 mmol) followed by HOBt (280 mg, 1.82 mmol), NH₄Cl (161 mg, 3.04 mmol) at 0° C. and stirred for 16 h at RT. After consumption of the starting material (by TLC), the reaction mixture was diluted with water (10 mL) and extracted with CH₂Cl₂ (2×30 mL). The combined organic layer was washed with citric acid solution (2×30 mL). The organic layer was dried over anhydrous Na₂SO₄, filtered and concentrated under reduced pressure. Obtained crude material was purified by silica gel column chromatography eluting 2% MeOH/DCM to afford compound (2S-FNL-2) (200 mg, 40%) as colorless liquid.

¹H-NMR: (500 MHz, DMSO-d₆): δ 7.53 (s, 2H), 4.59 (s, 1H), 4.02 (s, 1H), 3.77-3.70 (m, 2H), 3.62-3.53 (m, 2H), 3.46-3.33 (m, 1H), 2.17-2.03 (m, 2H), 1.88-1.71 (m, 2H), 1.38 (s, 9H), 1.18 (d, J=6.5 Hz, 3H);

Mass (ESI): 328.3 [M⁺+1]

### Synthesis of (2S, 3R)-3-hydroxy-2-(1-oxo-2, 5-diazaspiro [3.4] octan-2-yl) butanamide (2S-FNL-3)

To a stirring solution of compound (2S-FNL-2) (200 mg, 0.61 mmol) in CH₂Cl₂ (5 mL) was added TFA (0.5 mL, 6.1 mmol) at 0° C. and stirred at RT for 3 h. After completion of reaction (by TLC), the reaction mixture was concentrated under reduced pressure to obtained crude compound which was triturated with n-pentane/diethylether (5 mL/5 mL) to afford compound (2S-FNL-3) (100 mg) as white solid (TFA salt).

¹H-NMR: (400 MHz, D₂O): δ 4.33-4.29 (m, 2H), 4.09 (d, 1H), 3.95 (d, 1H), 3.57-3.48 (m, 2H), 2.51-2.46 (m, 2H), 2.25-2.19 (m, 2H), 1.31 (d, 3H);

LCMS, m/z: 455 [2M⁺+1]

### Synthesis of (2S, 3R)-3-hydroxy-2-(5-isobutyryl-1-oxo-2, 5-diazaspiro [3.4] octan-2-yl) butanamide (NYX-2925)

To a stirring solution of (2S-FNL-3) (500 mg (crude), 2.20 mmol) in CH₂Cl₂ (10 mL) was added TEA (1 mL, 7.70 mmol) followed by isobutyryl chloride (256 mg, 2.42 mmol) at 0° C. and stirred for 16 h at RT. After consumption of the starting material (by TLC), the reaction mixture was diluted with water (10 mL) and extracted with CH₂Cl₂ (2×30 mL). The combined organic layer was washed with citric acid solution (2×30 mL). The organic layer was dried over anhydrous Na₂SO₄, filtered and concentrated under reduced pressure. Obtained crude material was purified by silica gel column chromatography eluting 2% MeOH/DCM to afford the two diastereomers of (2S-FNL-4) (100 mg, 15.2%) as white solid. The two 2S-FNL-4 diastereomers were separated by repetitive silica gel chromatography to provide NYX-2925.

¹H-NMR: (500 MHz, DMSO-d₆): δ 7.63 (br s, ex withD₂O, 1H), 7.18 (br s, ex withD₂O, 1H), 4.77 (d, J=4.0 Hz, ex withD₂O, 1H), 4.04-4.00 (m, 1H), 3.95 (d, J=6.5 Hz, 1H), 3.76 (d, J=5.5 Hz, 1H), 3.66-3.63 (m, 1H), 3.53 (q, J=8.0 Hz, 1H), 3.39 (d, J=5.5 Hz, 1H), 2.72 (septet, J=6.5 Hz, 1H), 2.14-2.05 (m, 2H), 1.92-1.86 (m, 2H), 1.10 (d, J=6.0 Hz, 3H), 1.02 (d, J=6.5 Hz, 3H), 0.99 (d, J=6.5 Hz, 3H).

Mass (ESI): 298.4 [M⁺+1].

### Example 2: A Single-Blind, Exploratory, Placebo-Controlled NYX-2925 in Subjects with Fibromyalgia

A randomized, single-blind, parallel-group, placebo-controlled, multiple-dose study was conducted to assess the efficacy and safety of NYX-2925 in subjects with fibromyalgia.

The study was to determine whether daily dosing with NYX-2925 20 mg or 200 mg changed the markers of central pain processing in subjects with fibromyalgia by evaluating changes in evoked pain, visual stimulation, functional magnetic resonance imaging (fMRI), resting state functional connectivity magnetic resonance imaging (rs-fcMRI) and proton magnetic resonance spectroscopy (¹H-MRS) on active drug versus placebo. More specifically, the study was to examine whether:
1) NYX-2925 attenuates pain-evoked and aversive visual stimulus-evoked brain activation vs. placebo;
2) NYX-2925 reduces resting state functional connectivity (rs-fcMRI) between the insula and the Default Mode Network vs. placebo;
3) NYX-2925 reduces combined glutamate plus glutamine levels within pro-nociceptive brain regions, such as the insula and anterior cingulate cortex vs. placebo.

The study was also to evaluate the safety of NYX-2925 in subjects with fibromyalgia Exploratory Analyses:

The study evaluated changes (in active vs. placebo treatment periods) in the following: Daily Pain scores using the Numeric Pain rating Scales (NPRS); Brief Pain Inventory; Patient Reported Outcome Measurement Information System Fibromyalgia (PROMIS_{FM}) fatigue profile; Multidimensional Inventory of Subjective Cognitive Impairment (MISCI); PROMIS Sleep Disturbance short form; Revised Fibromyalgia Impact Questionnaire (FIQR); PROMIS Physical Disturbance short form; Hospital Anxiety and Depression Scale (HADS); Patient Global Impression of Change (PGI-C); the Fibromyalgia Survey Questionnaire; and Experimental pain sensitivity as assessed by Quantitative Sensory Testing (QST).

### Study Design

The timeline of events in the study are depicted in FIG. 1A. The study included up to a 30 day screening period, to confirm the diagnosis, assess eligibility, and complete the baseline magnetic resonance imaging (MRI). This was a single-blind, exploratory, placebo-controlled pilot study in which eligible subjects received 2 weeks of daily oral (QD) placebo, 2 weeks of NYX-2925 20 mg PO daily (low dose), 2 weeks of NYX-2925 200 mg PO daily (high dose), followed by a 1 week follow-up period. An MRI was conducted during the screening period, during the last week of the placebo period, during the last week of the NYX-2925 20 mg administration period, and during the last week of the NYX-2925 200 mg administration period. Study visits occurred during screening, then at day one and weeks 1, 2, 3, 4, 5, 6, and 7. An optional MRI was completed at the end of the follow-up period for consenting subjects. Study participants and study staff responsible for interpreting MRI results were blinded to the study drug administration.

Study drug (NYX-2925 or matching placebo) was dispensed to subjects for self-administration. Subjects recorded daily pain scores for the entire duration of the study. Other exploratory scales were completed during screening, after placebo, after NYX-2925 20 mg administration, after NYX-2925 200 mg administration, and after the follow-up period.

Adverse events (AEs), vital signs, electrocardiograms, physical examinations, Columbia Suicide Rating Scales, and clinical laboratory samples were collected throughout the study.

### Diagnosis and Main Criteria for Inclusion

Inclusion Criteria:
Female subjects 18-70 years of age.
Subject meets the 2010 American college of Rheumatology criteria for fibromyalgia.
Self reported clinical pain >4 on the Numeric Pain Rating Scale (NPRS) at screening and baseline, and consistent pain score collection during screening.
Subject receives and agrees to remain on their stable fibromyalgia treatment plan established at least 14 days prior to dosing. (Stable is defined as treatment with medication(s) and/or intervention(s) at the same prescribed dose).
Subject agrees to use only nonsteroidal anti-inflammatory (NSAID) or acetaminophen treatment as needed for breakthrough pain, and/or protocol specified medications for sleep (if needed).
Right handed.
Creatinine clearance >60 mL/min calculated by the Cockcroft Gault equation.
Female subjects of childbearing potential with a negative serum pregnancy test prior to entry into the study and who are practicing an adequate method of birth control (e.g., surgical sterilization, oral or parenteral contraceptives, intrauterine device that is considered safe for MRI procedures, barrier [condom and spermicide]) and who do not plan to become pregnant, breastfeed, or donate ova during the course of the study and for 28 days after the final administration of investigational product.
Ability to understand the requirements of the study, provide written informed consent, abide by the study restrictions, and agree to return for the required assessments.

### Exclusion Criteria:

Current or expected use of opioid or narcotic analgesics (other than tramadol), monoamine oxidase inhibitors (MAOIs), anticonvulsants (other than gabapentinoids), benzodiazepines, and sedatives, or hypnotics (with the exception of protocol specified allowed medications).
Unstable doses of allowed gabapentinoids, topiramate, tramadol, antidepressants, or muscle relaxants. Use of NSAIDs or acetaminophen 24 hours prior to imaging procedures is prohibited.
Pain due to current autoimmune or inflammatory disease such as rheumatoid arthritis, Ehlers-Danlos syndrome, systemic lupus erythematosus, inflammatory bowel disease, or other chronic widespread pain condition(s) that may confound fibromyalgia pain.
Untreated endocrine disorder that may confound fibromyalgia assessments.
Psychiatric or cognitive disorder (e.g., current schizophrenia, severe depression, suicidal ideation, dementia, etc.) that the investigator or sponsor considers significant for this-study.
Clinically significant alcohol or other substance abuse within the last 2 years, in the opinion of the investigator.
Positive screen for medically inappropriate or illegal use of drugs of abuse including benzodiazepines, opiates, cocaine, cannabinoids, and amphetamines.
Current treatment with N-methyl-D-aspartate receptor (NMDAR) ligands including ketamine, amantadine, dextromethorphan, memantine, methadone, dextropropoxyphene, or ketobemidone.
History of allergy, sensitivity, or intolerance to NMDAR ligands including ketamine, amantadine, dextromethorphan, memantine, methadone, dextropropoxyphene, or ketobemidone.
Women who are pregnant, breastfeeding, or planning to become pregnant or donate ova during the course of the study, and for 28 days after the final administration of investigational product.
Any impairment, activity, or situation that in the opinion of the investigator would prevent satisfactory completion of the of the study protocol. This includes unreliable or inconsistent pain scores, or an inability to tolerate the MRI procedure during screening. Huntington's, Parkinson's, Alzheimer's, multiple sclerosis, or a history of seizures, epilepsy, or strokes.
Contraindications to fMRI procedures. These may include but are not limited to: surgical clips, surgical staples, metal implants, and certain metallic dental material
Current or habitual use (within the last 12 months) of artificial nails, nails enhancements, or nail extension that cover any portion of either thumbnail, Exceptions include brief and/or occasional use, may be permissible at the discretion of the investigator
Abnormal laboratory results, medical history, or concurrent conditions that, in the opinion of the investigator or sponsor designated medical monitor, would preclude safe study participation, or interfere with study procedures/assessments.
Impaired hepatic function such as a known diagnosis of chronic liver disease or any abnormal serum total bilirubin, alanine transaminase (ALT), aspartate transaminase (AST), or alkaline phosphatase level deemed clinically significant by the investigator or sponsor at screening.
Subjects with history of sever renal impairment defined by renal dialysis or peritoneal dialysis, or who have undergone renal transplant.
Known history of significant cardiovascular condition such as myocardial infarction or congestive heart failure, or evidence of current uncontrolled cardiac arrhythmias, angina, or electrocardiographic evidence of acute ischemia, or QTcF>470 msec, or uncontrolled hypertension or blood pressure characterized by resting systolic blood pressure >140 mmHg or resting diastolic >90 mmHg.
Current evidence dysplasia or history of malignancy (including lymphoma and leukemia) in the last 5 years, with the exception of successfully treated non-metastatic basal cell or squamous cell carcinoma of the skin or localized carcinoma in situ of the cervix.
Human immunodeficiency virus (HIV) infection, hepatitis, or other ongoing infectious disease that the investigator considers clinically significant.
History of sever renal or hepatic impairment, in the opinion of the investigator or the sponsor designated medical monitor.
History of photosensitive migraine or migraine with aura, which in the opinion of the investigator would be contraindicated in MRI procedures.
History of lower limb vascular surgery or current lower limb vascular dysfunction that would interfere with MRI procedures, in the opinion of the investigator.
Received an investigational drug or device within 30 days (or 5 half-lives, whichever is longer) of dosing.
Previous treatment with NYX-2925.
Resting heart rate <45 or >95 beats per minute.

### Investigational Product, Dosage, and Mode of Administration:

NYX-2925 is a small molecule ((2S, 3R)-3-hydroxy-2-((R)-5-isobutyryl-1-oxo-2,5-diazaspiro[3.4]octan-2-yl)butanamide), provided as capsules for PO administration in strengths of 10 and 100 mg NYX-2925 per capsule.

The PO study drug comprises inert United States Pharmacopeia-grade excipients in a capsule made of hydroxyl-propyl cellulose; the formulation comprises a dry blend of NYX-2925; microcrystalline cellulose, National Formulary; pregelatinized starch, National Formulary; and magnesium stearate United States Pharmacopeia.

The placebo capsules matched to NYX-2925 contained only the inactive ingredients listed previously.
NYX-2925, 10 and 100 mg oral capsules
NYX-2925 low dose: 20 mg (two 10 mg capsules) PO QD for 2 weeks
NYX-2925 high dose: 200 mg (two 100 mg capsules) PO QD for 2 weeks

### Duration of Treatment/Study (Up to Approximately 11 Weeks)

Screening period—up to 30 days.
Placebo once daily for 2 weeks.
NYX-2925 for 4 weeks.
Follow-up period for 1 week.

### Reference Therapy, Dosage and Mode of Administration

Placebo, oral capsule(s)
2 placebo capsules PO QD for 2 weeks.

### Prior and Concomitant Therapy

For the duration of the study, subjects must have agreed to remain on their stable fibromyalgia treatment plan established prior to study participation. Stable was defined as treatment with medication(s) and/or intervention(s) at the same prescribed dose established at least 14 days prior to study drug dosing.

The use of opioid or narcotic analgesics (other than tramadol), monoamine oxidase inhibitors, anticonvulsants (other than gabapentinoids), sedatives, benzodiazepines, and hypnotics (unless specified as an allowed medication) was prohibited. Use of NMDAR ligands, including ketamine, amantadine, dextromethorphan, memantine, methadone, dextropropoxyphene, or ketobemidone, was prohibited. In addition, unstable doses of antidepressants or muscle relaxants were prohibited.

Subjects who were using tricyclic antidepressants, gabapentinoids, tramadol, or topiramate must have been using a stable dose for at least 2 months prior to dosing.

Breakthrough pain could have been treated with NSAIDs or acetaminophen; however, use of NSAIDs or acetaminophen 24 hours prior to visits that included MRI was prohibited.

Zopiclone, zolpidem, zaleplon, eszopiclone, ramelteon, melatonin, suvorexant, and trazodone could have been taken at a stable dose to aid sleep. Prescribed marijuana use was permitted if it was part of the subject's stable fibromyalgia treatment plan. Washout from excluded medication was allowed only if the Investigator deemed it was medically appropriate. The washout period must have covered 5 half-lives of the drug and should have been completed during the Screening Period. The Investigator was encouraged to contact the Sponsor with any questions related to excluded, washout, unanticipated prohibited, and/or concomitant medications.

### Criteria for Evaluation

PS Efficacy:
Evoked fMRI findings: decreased activation of pronociceptive brain activity
Rs-fcMRI findings: decreased Default Mode Network—insula connectivity
¹H-MRS findings: reduced insular glutamate+glutamine (Glx)

### Safety:

Adverse events
Vital sign measurements
Clinical laboratory test results
Electrocardiogram results
Physical examination findings
Columbia-Suicide Severity Rating Scale (C-SSRS)

### Exploratory Analyses:

Numeric Pain Rating Scale
Brief Pain Inventory—Short Form
Revised Fibromyalgia Impact Questionnaire
Patient Reported Outcomes Measurement Information System Fibromyalgia (PROMIS_{FM})
Multidimensional Inventory of Subjective Cognitive Impairment
PROMIS Sleep Disturbance short form
PROMIS Physical Disturbance short form
Hospital Anxiety and Depression Scale
Patient Global Impression of Change
Fibromyalgia Survey Questionnaire
Quantitative Sensory Testing (pressure pain sensitivity, cuff algometry, visual stimulation task)
   An optional MRI was completed during the follow-up period to assess duration of effect.

### Subjects for Study

Twenty-two female subjects with fibromyalgia were enrolled into the study and 20 subjects completed the study. The subject that did not complete the study did not complete the Follow-Up Visit.

Results of Study of NYX-2925 in Patients with Fibromyalgia

Pharmacodynamics

Multiple effects of NYX-2925 were observed on Glx levels acquired during ¹H-MRS. NYX-2925 reduced Glx levels in the dACC at rest and the posterior insula following experimental pressure pain. Higher pre-treatment Glx levels were also associated with subsequent increases in pressure pain thresholds following NYX-2925 20 mg. The relationships between Glx and QST-derived pain thresholds were opposite during NYX-2925 200 mg as compared to pre-treatment timepoints.

NYX-2925 also had effects on brain connectivity patterns, showing decreases in connectivity to specific regions in the dACC, pgACC, thalamus, and medial prefrontal cortex. Of note some of the regional brain areas showing decreased connectivity, also showed effects of NYX-2925 on Glx. One region in particular was the dACC. This region displayed marked decreases in connectivity to the precuneus (a region of the Default Mode Network) and this change in connectivity was associated with decreases in pain interference. These findings suggest that NYX-2925 induces changes in brain functional connectivity.

Efficacy

Several exploratory analyses were performed on the clinical scales data. No adjustments were made for multiple comparisons. Nonetheless, nominally statistically significant improvements were observed from Week 2 (placebo) to Week 4 (NYX-2925 200 mg) for the following efficacy endpoints:
NPRS average pain score, with mean change of −0.66 (p=0.0072, paired t-test)
NPRS worst pain score, with mean change of −0.61 (p=0.0360, paired t-test)
PROMIS_{FM} Total Score, with mean change of −5.6 (p=0.0049, paired t-test)
PROMIS_{FM} Experience Subdomain Score, mean change of −2.2 (p=0.0036, paired t-test)
PROMIS_{FM} Social Impact Subdomain Score, with mean change of −1.4 (p=0.0329, paired t-test)
PROMIS_{FM} Motivational Impact Subdomain Score, with mean change of −1.0 (p=0.0490, paired t-test)
Revised Fibromyalgia Impact Questionnaire Symptoms Domain, with mean change of −6.2 (p=0.0207, paired t-test)
Revised Fibromyalgia Impact Questionnaire Total score, with mean change of −6.3 (p=0.0135, paired t-test)

Mean reductions in the BPI-SF Pain Severity Index and Pain Interference Index scores from Week 2 (placebo) to Week 6 (NYX-2925 200 mg) were −0.27 and −0.66, respectively. A mean improvement was also observed for the Multidimensional Inventory of Subject Cognitive Impairment Total score from Week 2 (placebo) to Week 6 (NYX-2925 200 mg). These findings were not statistically significant.

Smaller mean improvements from Week 2 (placebo) were generally observed following treatment with NYX-2925 20 mg (Week 4), showing a positive trend for treatment with NYX-2925 20 mg.

In summary, consistent improvement following treatment with NYX-2925 was observed across measures of pain and quality of life in subjects with fibromyalgia.

### Overall Conclusions

In this study, NYX-2925 demonstrated antinociceptive activity in neuroimaging evaluations.

Clinically meaningful and statistically significant improvements were also observed in pain, fatigue, the overall intensity of fibromyalgia symptoms, and their impact on function.

NYX-2925 was safe and well tolerated in this study with no discontinuations due to treatment-emergent adverse events (TEAE). No serious adverse events were reported.

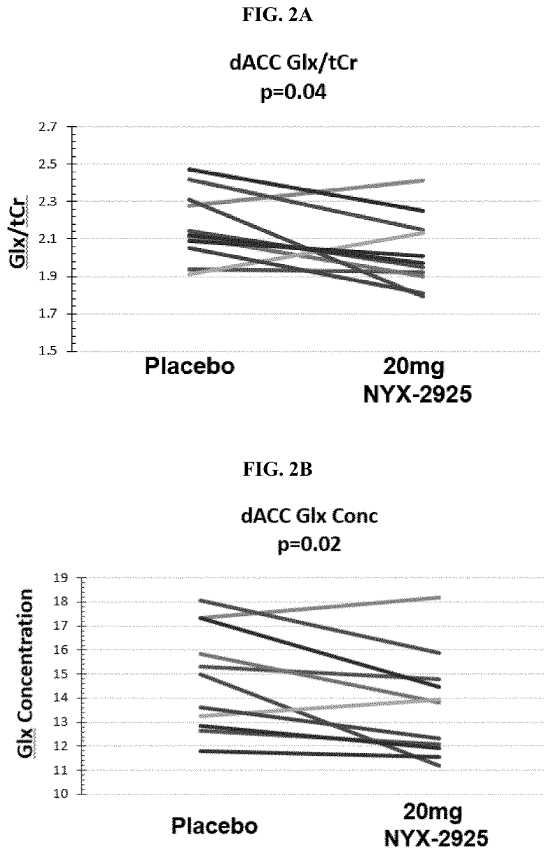

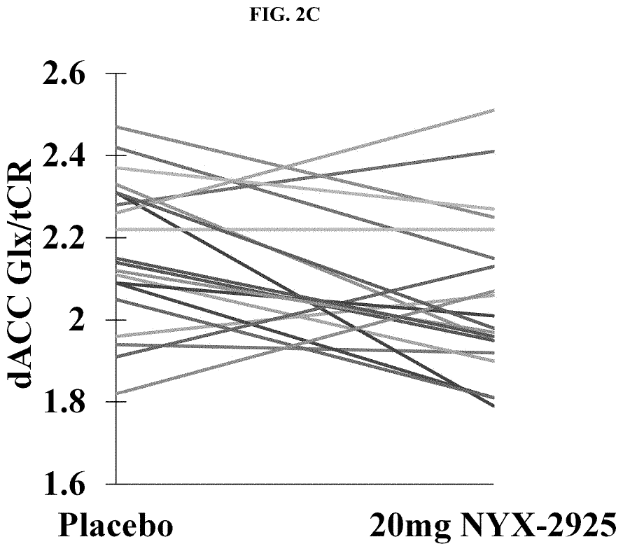

Fibromyalgia is associated with increased overall levels of combined glutamate and glutamine (Glx) and prior studies have shown a correlation between pain severity and higher Glx levels in certain brain regions. Compared to placebo, administration of NYX-2925 resulted in statistically significant reductions of Glx levels in these key pain-regulating brain regions, including the dorsal anterior cingulate cortex (dACC) at rest (p<0.05), as shown in FIGS. 2A, 2B and 2C. The lines in FIGS. 2A, 2B and 2C each represent an individual patient, and show reduction in resting Glx levels for the 20 mg dose of NYX-2925 vs. placebo. No significant decrease was seen in the dACC Glx levels for the 200 mg dose.

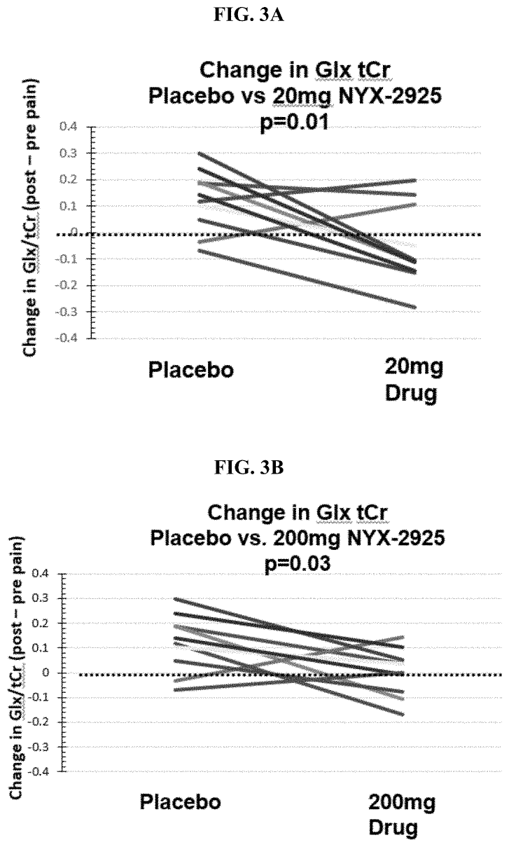

Compared to placebo, administration of NYX-2925 also resulted in statistically significant reductions of increases in Glx levels in the posterior insular cortex following an evoked pain stimulus (p<0.05), as shown in FIGS. 3A and 3B. The lines in FIGS. 3A and 3B each represent an individual patient, and show reduction in pain related Glx/tCr level increases for both the 20 mg and 200 mg doses of NYX-2925 vs. placebo. NYX-2925 administration also resulted in various connectivity changes that are known to be associated with central pain processing. The observed changes in objectively measured Glx levels and altered connectivity patterns correlated with improvements on patient-reported outcomes.

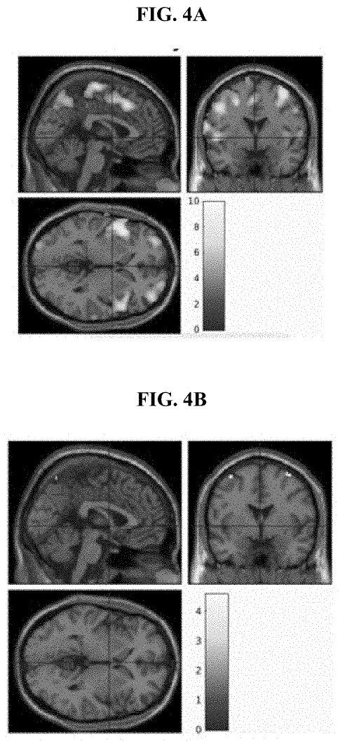

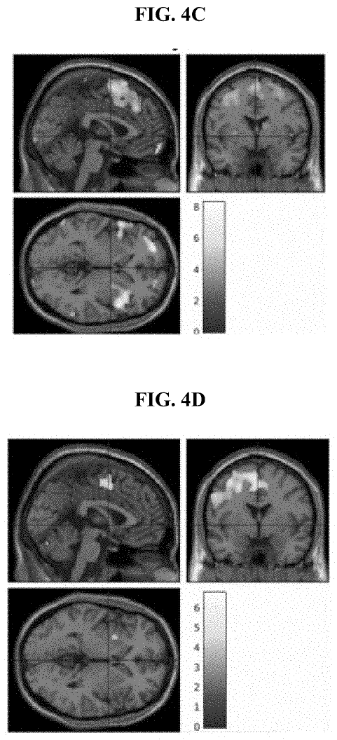

FIGS. 4A-4D illustrate induced changes in pain-evoked brain activity as seen in brain imaging. FIG. 4A depicts pain-evoked brain activity in subject 1 after placebo, and shows several lighter colored areas indicating brain activity. FIG. 4B depicts pain-evoked brain activity in subject 1 after a 200 mg dose of NYX-2925, and shows essentially no lighter colored areas indicating brain activity, thus showing a reduced pain-related brain activity after NYX-2925 dosing. Similarly, FIGS. 4C and 4D depict pain-evoked brain activity after dosing subject 2 with placebo and NYX-2925 respectively, and likewise show a reduction in pain-related brain activity in FIG. 4D after NYX-2925 dosing.

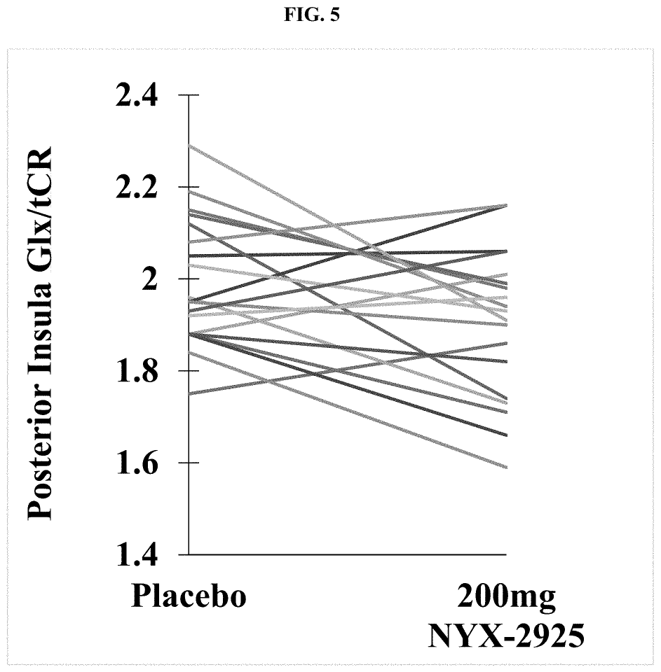

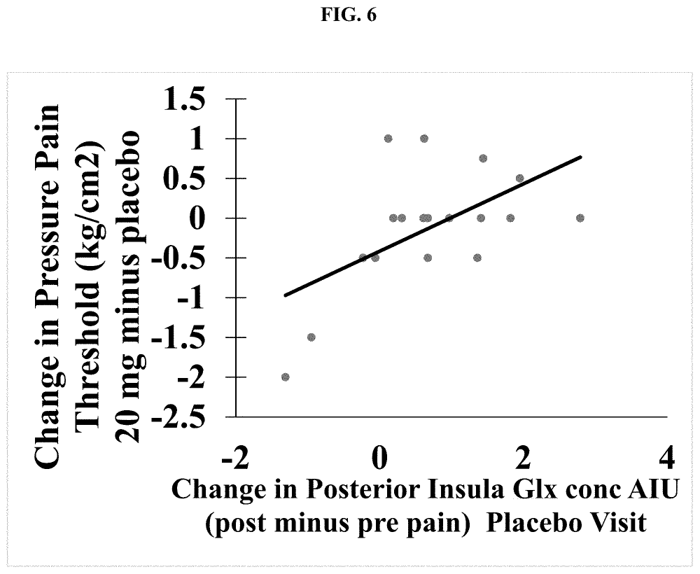

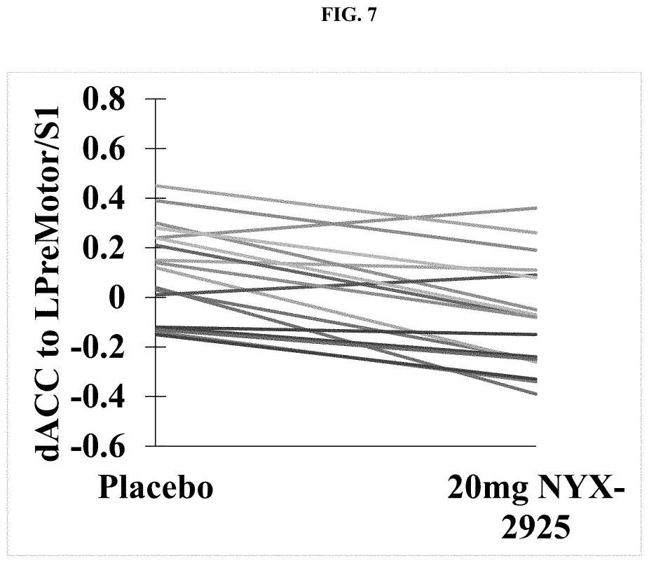

NYX-2925 was shown to significantly reduce resting posterior insula Glx levels following evoked pain (by cuff pressure applied to the left calf). See FIG. 5. Greater concentration of pain-evoked Glx in posterior insula at baseline was associated with greater reductions in pain sensitivity following treatment. See FIG. 6. NYX-2925 (20 mg.) administration resulted in reduced connectivity between pain-related brain regions, including connectivity between dACC and primary somatosensory cortex. See FIG. 7. Regarding patient-reported outcomes, significant clinical improvements on key symptoms of fibromyalgia were observed following treatment with NYX-2925. These patient-reported outcomes included average daily pain and worst daily pain measured using the 10-point Numeric Rating Scale (NRS), the impact of patients' fibromyalgia on daily living measured by the Revised Fibromyalgia Impact Questionnaire (FIQR), scores on the Patient Reported Outcomes Measurement Information System Fibromyalgia (PROMIS_{FM}) scale, pain severity and impact on functioning measured by the Brief Pain Inventory (BPI), mood and anxiety measured by the Hospital Anxiety and Depression Scale (HADS), and cognitive impairment measured using the Multidimensional Inventory of Subjective Cognitive Impairment (MISCI).

At baseline, patients in the study had a mean average daily pain score of 5.3 on the NRS, which has a range from 0 to 10 (where 0=no pain and 10=worst pain imaginable) and had a mean baseline total FIQR score of 54.4 (this scale has a maximum score of 100, with higher scores indicating worse fibromyalgia). Based on these scores, the patients in the study were considered to have moderate-to-severe fibromyalgia.

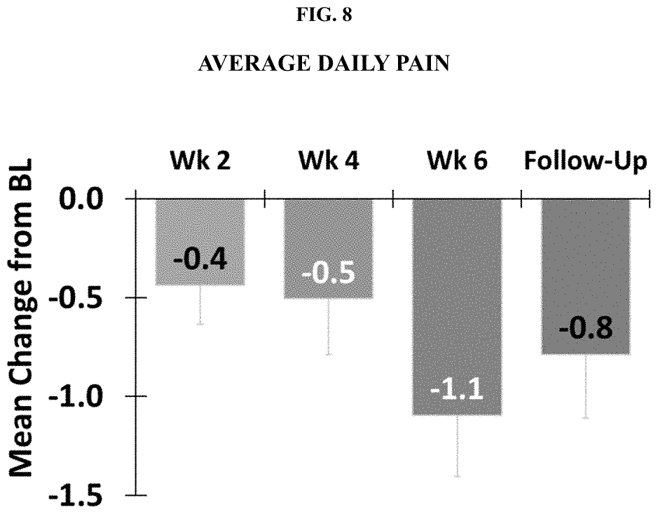

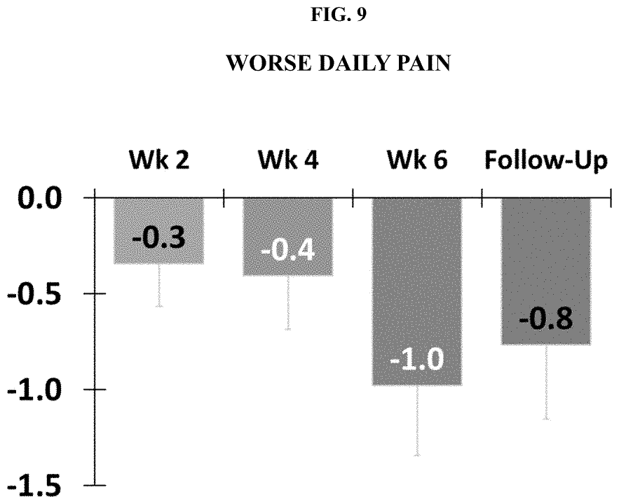

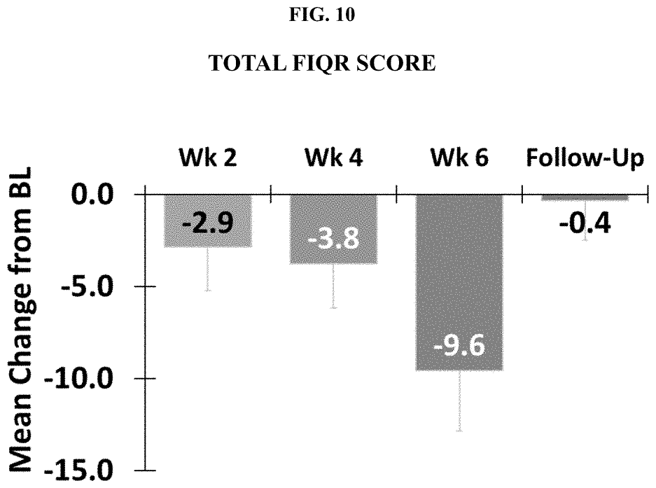

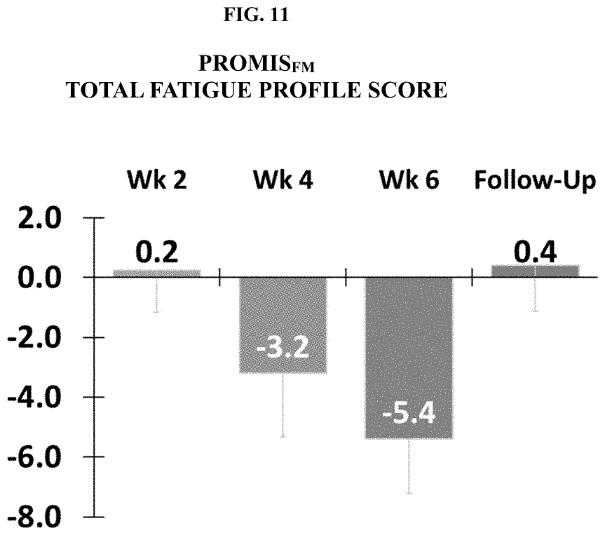

Among other clinical measures, at week six (i.e., after four weeks of NYX-2925 administration), NYX-2925 resulted in statistically significant improvements in:
Average daily pain score: 1.1-point reduction from baseline (n=19, p<0.01 vs. baseline, p<0.01 vs. placebo) on a scale ranging from 0 to 10 (see FIG. 8)
Worst daily pain score: 1.0-point reduction from baseline (n=19, p<0.05 vs. baseline, p<0.05 vs. placebo) on a scale ranging from 0 to 10 (see FIG. 9)
Total FIQR score: 9.6-point reduction from baseline (n=21, p<0.01 vs. baseline, p<0.05 vs. placebo) on a scale ranging from 0 to 100 (see FIG. 10)
PROMIS_{FM} Fatigue Profile total score: 5.4-point reduction from baseline (n=21, p<0.01 vs. baseline, p<0.01 vs. placebo) on a scale ranging from 16 to 80 (see FIG. 11)

In addition, trends of improvement were observed on the other clinical measures evaluated in the study, demonstrating improvement in fibromyalgia symptoms broadly. For example, reductions from baseline for average daily pain, worse daily pain, total FIQR score, and PROMIS_{FM} total fatigue profile score were observed for NYX-2925 20 mg. Accordingly, clinically meaningful and statistically significant improvements were observed following treatment with NYX-2925 compared to baseline and placebo (Week 2) for pain and quality of life measures.

### Dorsal Anterior Cingulate Cortex

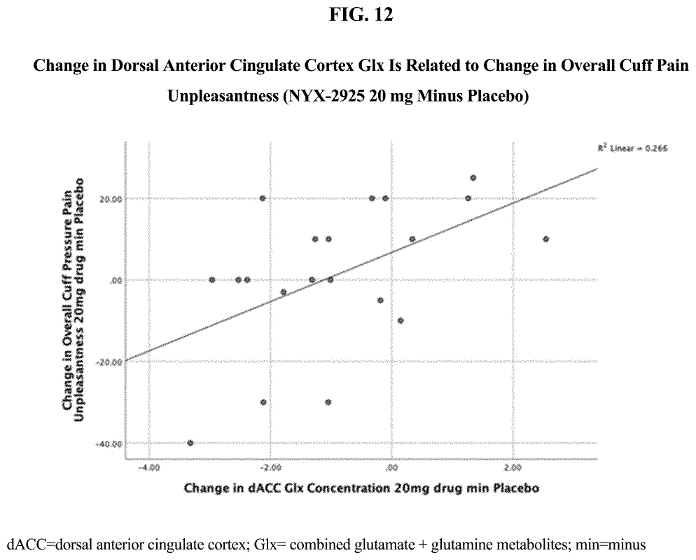

The dACC showed decreased resting Glx (concentrations and ratios to creatine; both p<0.05) with NYX-2925 20 mg. Decreases in Glx in dACC from placebo to NYX-2925 20 mg were associated with decreased cuff pain unpleasantness during scanning visit from placebo to NYX-2925 20 mg (see FIG. 12).

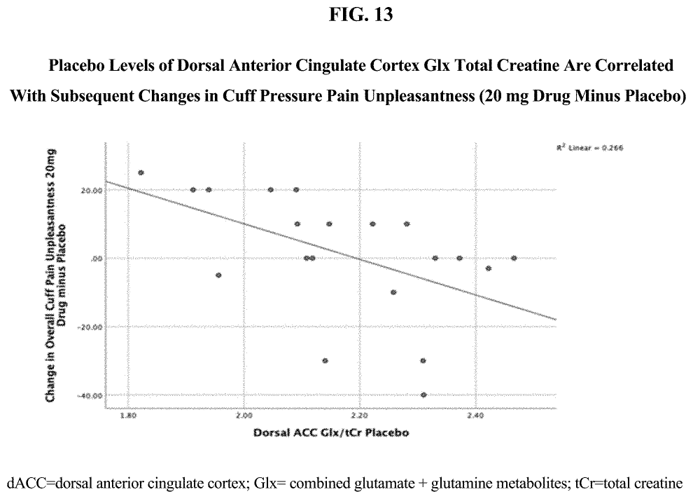

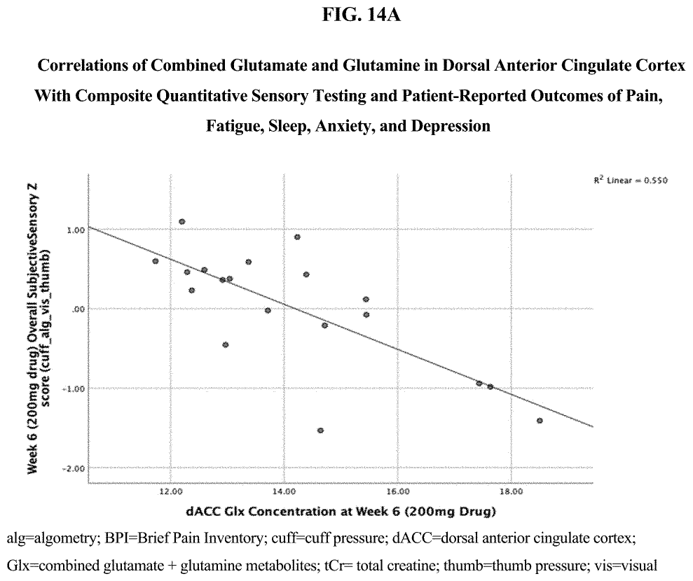

Placebo and baseline dACC Glx levels (ratios only) predicted change in cuff unpleasantness following NYX-2925 20 mg (see FIG. 13; p<0.005). Higher dACC Glx at placebo and baseline were related to greater decreases in cuff pain unpleasantness.

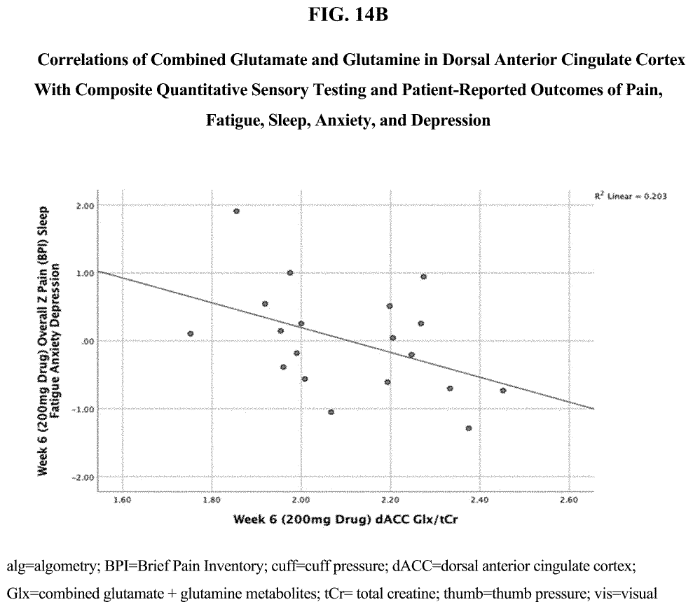

No effect of NYX-2925 200 mg was observed on mean Glx levels in the dACC; however, at NYX-2925 200 mg, higher Glx (concentrations and ratios to creatine; both p<0.05) were associated with less pain on the composite QST measure (combined Z-score across MAST, algometry, cuff, visual; p<0.0005; FIG. 14A) and less patient-reported outcomes (combined Z-score of pain, fatigue, sleep, anxiety, and depression; p<0.05; FIG. 14B). Moreover, increases in dACC Glx (ratios to creatine and trending for concentrations) from NYX-2925 20 mg to 200 mg were associated with less QST pain (Z-score; p=0.05) and less patient-reported symptoms (Z-score; p<0.05).

### Posterior Insula

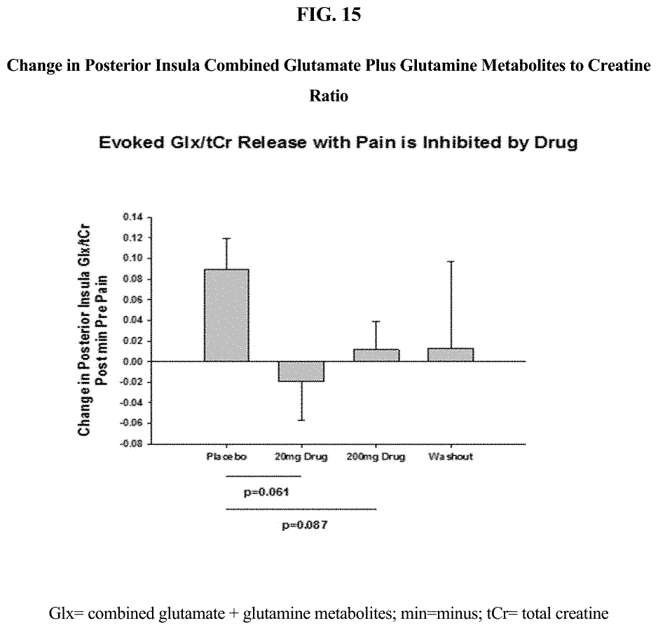

At the placebo visit, evoked cuff pain increased posterior insula Glx levels (concentrations and ratios; both p<0.05). These pain-evoked increases in Glx were not significant at the NYX-2925 20 mg or 200 mg visits (all p>0.60). When compared to the placebo visit, there was a trend for NYX-2925 20 mg and 200 mg to reduce evoked pain Glx levels (p<0.10; FIG. 15). Compared to placebo, there was a significant decrease in Glx levels (ratios to creatine only) post-pain on NYX-2925 200 mg (p<0.05).

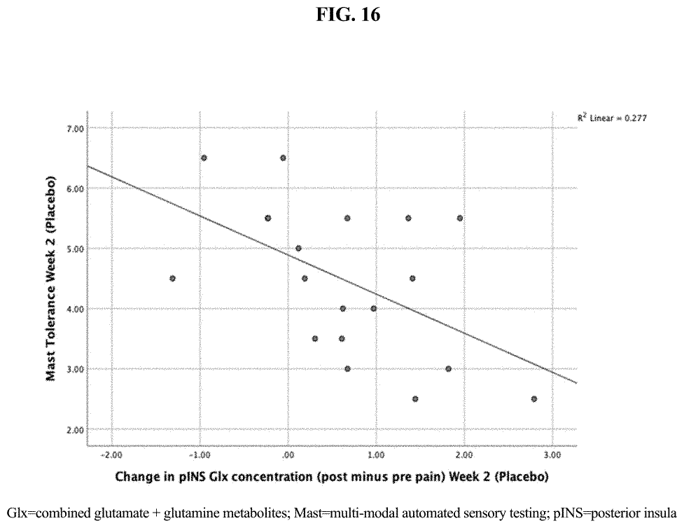

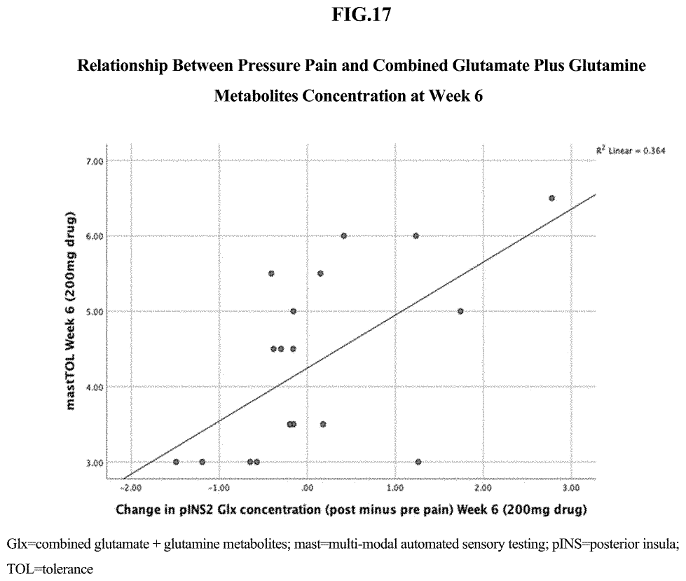

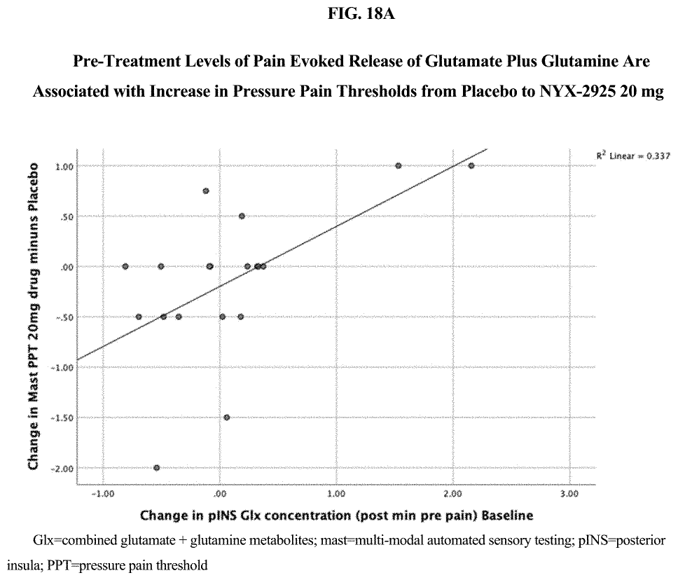

Glx release at the placebo visit was related to decreased pressure pain thresholds at the thumb using QST (FIG. 16). This is very similar to Harris 2009, where fibromyalgia patients with greater Glx were more sensitive to pressure pain (for n=19 and 20 samples).

However, evoked pain release of Glx at the NYX-2925 200 mg visit is opposite of the placebo visit. Treatment with NYX-2925 200 mg, more Glx release (concentrations only) is related to higher pressure pain thresholds (FIG. 17).

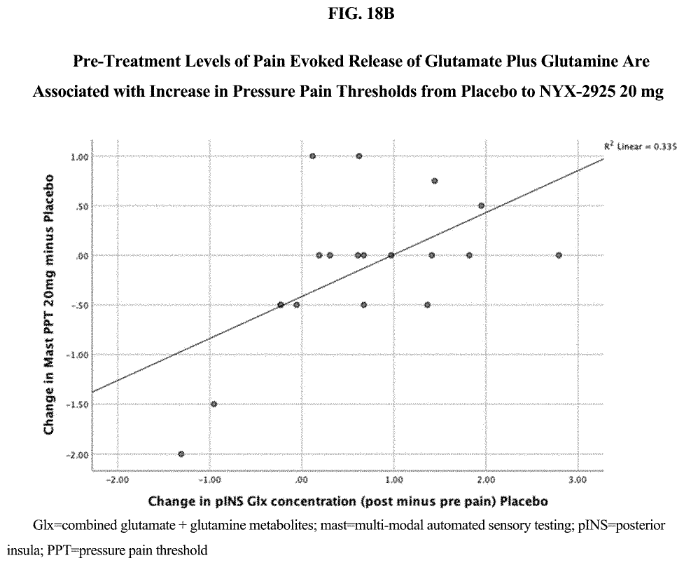

Higher placebo and baseline levels of Glx (concentrations) during evoked pain were correlated with increases in pressure pain thresholds from placebo to NYX-2925 20 mg (FIGS. 18A and 18B, respectively). Greater glutamate+glutamine metabolites release pre-drug was associated with subsequent increases in pressure pain thresholds with NYX-2925 20 mg.

Regression models showed that increased Glx concentration release following pain (at both baseline and placebo visits) was associated with increased pressure pain thresholds on NYX-2925 20 mg, while controlling for placebo and baseline pressure pain thresholds and age.

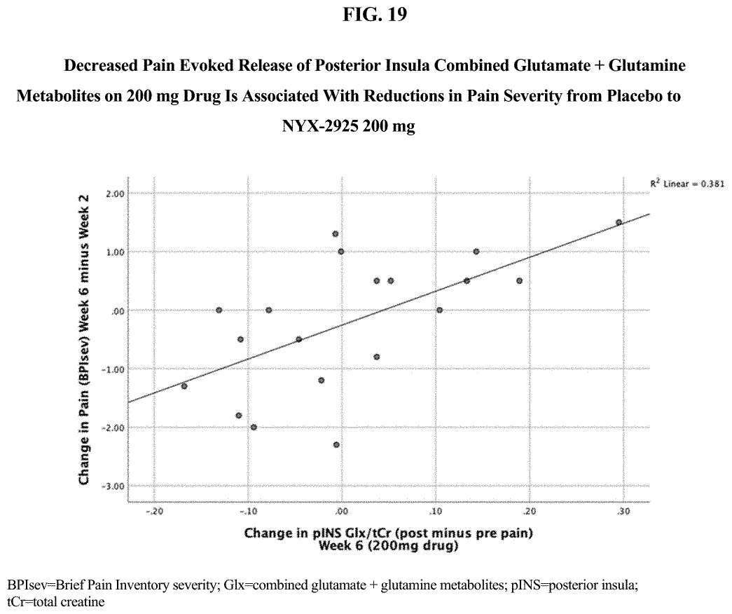

Change in posterior insula Glx total creatine following evoked pain on NYX-2925 200 mg, was positively correlated with change in BPI-SF and Fatigue from NYX-2925 200 mg minus placebo for the analysis adjusted for age. Less Glx release on NYX-2925 200 mg was related to greater pain and fatigue improvement with NYX-2925 200 mg (FIG. 19 shows correlation between change in posterior insula Glx total creatine versus change in BPI-SF severity).

### Functional Magnetic Resonance Imaging

Several analyses were conducted with the fMRI resting state and task data. Results were assessed by: 1) a primary analysis, where a finding was deemed significant with a corrected cluster level p<0.05, derived from an uncorrected voxel-wise p<0.001, and 2) an exploratory analysis, where a finding was deemed significant with an uncorrected voxel-wise p<0.005 and having a cluster greater than or equal to 80 contiguous voxels. Results that met significance in the primary analyses had connectivity values extracted and imported into SPSS v25 where paired t-tests were performed to assess for potential outliers contributing to the finding. Remaining data were then correlated with clinical measures using parametric bivariate Pearson correlations. Results were deemed significant at p<0.05.

### Resting State: Group Independent Component Analysis

Group ICA was performed (n=18) for the resting state scan, and the network components for the Default Mode Network, the Salience Network, and the Dorsal Attention Network were computed. No results met significance for the primary analysis with the Default Mode Network. With the exploratory analyses, resting state connectivity was decreased from the placebo visit to the NYX-2925 200 mg visit (placebo >NYX-2925 200 mg) between the Default Mode Network and the left mid cingulate cortex. This exploratory finding is consistent with previous studies that report decreased functional connectivity between the Default Mode Network and brain regions involved in nociception. No results met significance for the primary Salience Network analysis. In exploratory analyses, resting state functional connectivity was decreased from the placebo visit to the NYX-2925 200 mg visit (placebo >NYX-2925 200 mg) between the Salience Network and the right putamen/thalamus. Here, augmented connectivity between Salience Network, a network comprised of pronociceptive regions, and putamen and thalamus, regions also involved in nociception, was decreased following treatment with NYX-2925. There were no significant results with primary and exploratory analyses with the Dorsal Attention Network.

### Resting State: Seed-Based Region of Interest Analysis

Seed-based, region of interest, functional connectivity analyses were performed (n=18) for regions that were used to acquire ¹H-MRS data: dACC, perigenual anterior cingulate cortex (pgACC), right anterior insula cortex, right posterior insula cortex, precuneus, thalamus, and medial prefrontal cortex.

### Dorsal Anterior Cingulate Cortex

Primary analyses showed decreased functional connectivity between the dACC seed and several brain regions from the placebo visit to the NYX-2925 20 mg visit (placebo >NYX-2925 20 mg). These decreases were found between dACC seed and a) right superior temporal gyms (family-wise error [PWE] P_{FWE}=0.031), b) left premotor cortex/primary somatosensory cortex (P_{FWE}=0.030), c) right precuneus (P_{FWE}=0.031), and d) left precentral gyrus/premotor cortex (P_{FWE}=0.049). While not meeting cluster level correction significance, there was also decreased connectivity between the dACC seed and the right posterior insula cortex. These results are consistent with previous reports showing decreased connectivity between a pronociceptive seed regions, such as the dACC, and other brain regions that are thought to be pronociceptive (ie, superior temporal gyms, primary somatosensory cortex, posterior insula), as well as a decoupling of this pronociceptive seed and the precuneus, a region involved in the Default Mode Network.

### Perigenual Anterior Cingulate Cortex

Resting state functional connectivity was decreased from the placebo visit to the NYX-2925 20 mg visit (placebo >NYX-2925 20 mg) between the antinociceptive pgACC seed and the left precuneus/precentral gyms (P_{FWE}<0.00001), and from the placebo visit to the NYX-2925 200 mg visit (placebo >NYX-2925 200 mg) between the same seed and the right inferior frontal gyms (P_{FWE}=0.008).

### Right Anterior Insula Cortex

Resting state connectivity was decreased from the baseline visit to the NYX-2925 200 mg visit (baseline >NYX-2925 200 mg) between 2 pronociceptive regions: the right anterior insula cortex seed and the right ventral anterior thalamic nucleus (P_{FWE}=0.018). This finding is in accordance with published findings showing that pregabalin decreases resting state functional connectivity between these regions.

### Right Posterior Insula Cortex

There were no significant findings for the right posterior insula cortex seed in the primary analyses. Exploratory analysis with this seed found a decrease in connectivity from the placebo visit to the NYX-2925 20 mg visit (placebo >NYX-2925 20 mg) to the dACC. Decreased resting state connectivity was again seen between 2 brain regions involved in nociceptive pain processing.

### Precuneus

There were no significant results with the precuneus seed for the primary analyses; however, there were several findings with the exploratory analyses. Precuneus resting state connectivity to right anterior insula and left dACC was decreased from the baseline visit to the NYX-2925 20 mg visit (baseline >NYX-2925 20 mg). Decreases in resting state functional connectivity were also found from the placebo visit to the NYX-2925 20 mg visit (placebo >NYX-2925 20 mg) between the precuneus and the right mid cingulate cortex. Finally, resting state functional connectivity was decreased from the placebo visit to the NYX-2925 200 mg visit (placebo >NYX-2925 200 mg) between the precuneus seed and the right posterior/mid cingulate cortex. These results indicate that NYX-2925 reduces connectivity between a key region of the Default Mode Network, the precuneus, and pronociceptive brain regions—consistent with previous studies showing a disentanglement between the Default Mode Network and other pain processing regions with analgesic treatment.

### Thalamus

In the primary analysis, there was a significant decrease in resting state connectivity from the baseline visit to the NYX-2925 200 mg visit (baseline >NYX-2925 200 mg) between the thalamus seed and the bilateral cuneus (P_{FWE}<0.00001). In exploratory analysis, there were decreases in connectivity from the placebo visit to the NYX-2925 200 mg visit (placebo >NYX-2925 200 mg) between the thalamus seed and the right anterior prefrontal cortex, as well as bilateral precuneus. These results all show a dissociation between the Default Mode Network and the thalamus, a nociceptive processing region following treatment with drug.

### Medial Prefrontal Cortex

Significant decreases in resting state connectivity for the primary analyses were found from the baseline visit to the NYX-2925 200 mg visit (baseline >NYX-2925 200 mg) between the medial prefrontal cortex seed and the right anterior prefrontal cortex/dorsolateral prefrontal cortex (P_{FWE}=0.001). Decreases were also observed from the placebo visit to the NYX-2925 200 mg visit (placebo >NYX-2925 200 mg) between the medial prefrontal cortex seed and the right anterior prefrontal cortex (P_{FWE}=0.003) and the right mid cingulate cortex (P_{FWE}=0.036). These results show decreases in functional connectivity between a region of the anterior Default Mode Network and other intrinsic Default Mode Network regions, as well as to both antinociceptive (dorsolateral prefrontal cortex) and pronociceptive regions (mid cingulate cortex).

### Evoked Cuff Pain Task Bold Activations—Pain50 Pressure >Rest Contrast

No significant BOLD activations were found for the evoked cuff pain task in primary analyses. In exploratory analyses, BOLD activations were decreased from the placebo visit to the NYX-2925 20 mg visit (placebo >NYX-2925 20 mg) within the right mid cingulate cortex and left primary somatosensory cortex/supplementary motor area. Further, BOLD activations were decreased from the placebo visit to the NYX-2925 200 mg visit (placebo >NYX-2925 200 mg) within the right putamen/insula cortex, bilateral primary somatosensory cortex/cingulate cortex, left putamen, and the left posterior insula cortex. These regions have been described previously as having augmented BOLD activation in chronic pain patients during evoked pain tasks, and here these activations were reduced after treatment with NYX-2925.

### Visual Task Bold Activations

No significant or marginal changes in BOLD activation were found for the visual task at either dose of drug relative to baseline or placebo.

Main Effect Resting State Functional Connectivity Correlations with Clinical Measures

Several significant results from the primary analyses were found to be correlated with clinical measures. In most cases, these analyses showed that decreased resting state functional connectivity following treatment between regions of the Default Mode Network and regions involved in pain processing was associated with improvements in clinical, or experimental, pain. Specific results and accompanying correlation graphs are included below.

### Dorsal Anterior Cingulate Cortex

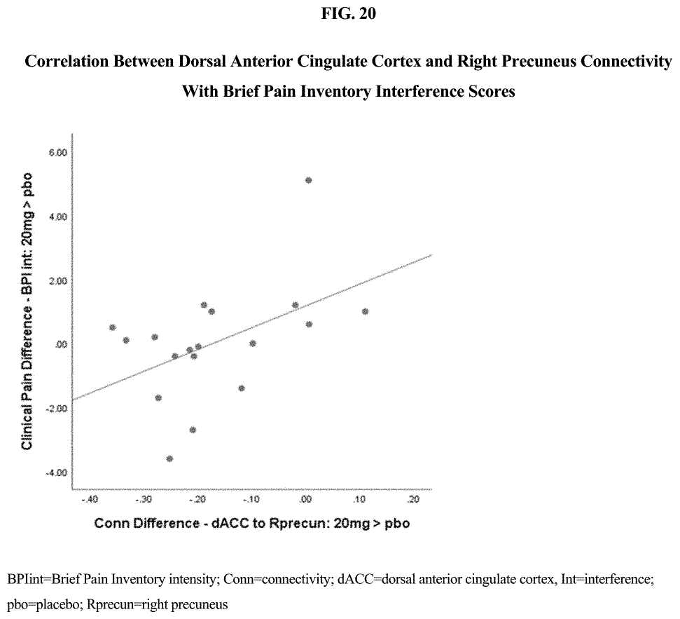

Decreased functional connectivity between the dACC and the right precuneus from the placebo visit to the NYX-2925 20 mg visit was found to be positively correlated with the change in BPI-SF interference scores over the same period (r=0.472, p=0.048, see FIG. 20). This indicates that decreased functional connectivity between these regions is associated with improvements in clinical pain interference following treatment with NYX-2925.

### Perigenual Anterior Cingulate Cortex

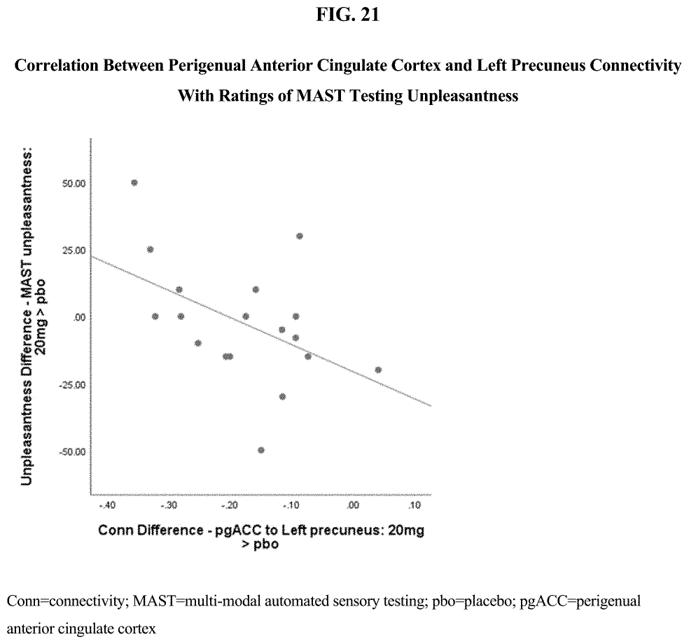

Functional connectivity changes between the pgACC and the left precuneus/postcentral gyms from the placebo visit to the NYX-2925 20 mg visit were found to be negatively associated with overall unpleasantness ratings from the evoked pressure pain MAST testing session (r=−0.476, p=0.046, see FIG. 21). This analysis suggests that increased functional connectivity between an antinociceptive seed and the precuneus correlates with less perceived unpleasantness of MAST testing. However, note that the main effect of NYX-2925 is a reduction in connectivity between these regions.

### Medial Prefrontal Cortex

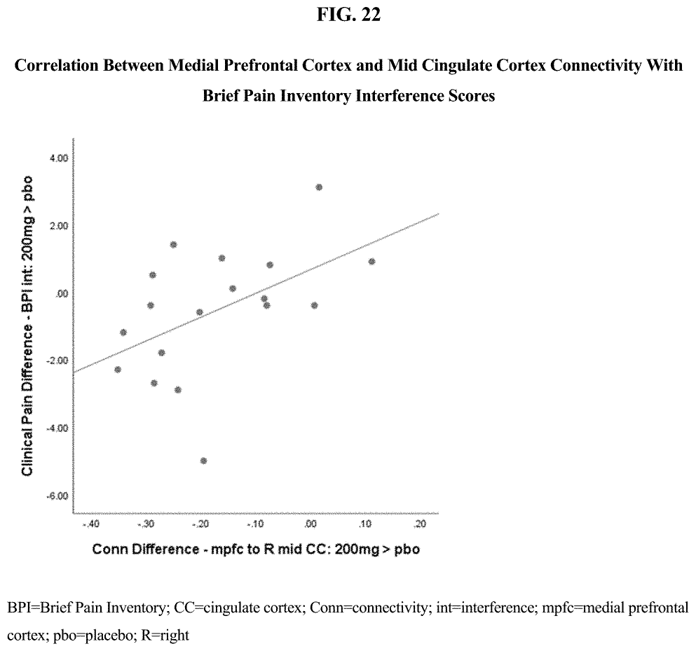

Decreased functional connectivity between the medial prefrontal cortex and the right mid cingulate cortex from the placebo visit to the NYX-2925 200 mg visit were positively correlated with changes in BPI-SF interference scores (r=0.496, p=0.036, see FIG. 22). This suggests that decreased resting state functional connectivity between a region in the Default Mode Network, the medial prefrontal cortex, and a pronociceptive region, the right midcingulate cortex, correlates with improvement in clinical pain interference.

### Thalamus

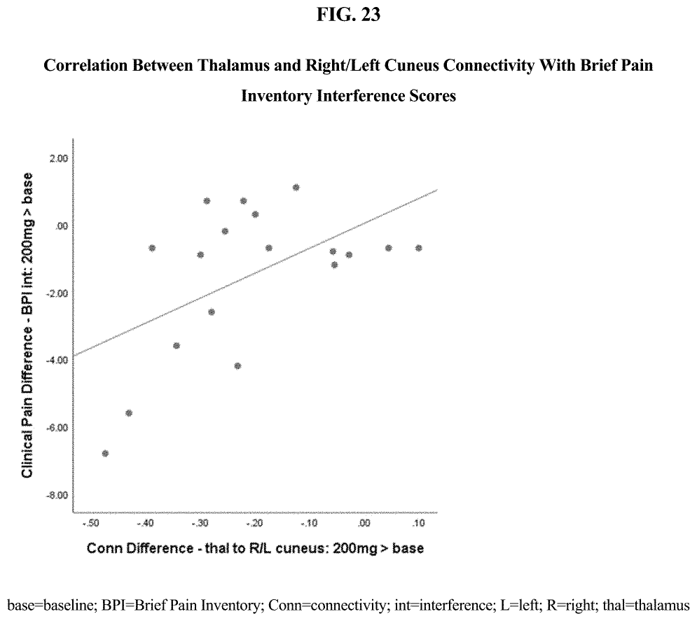

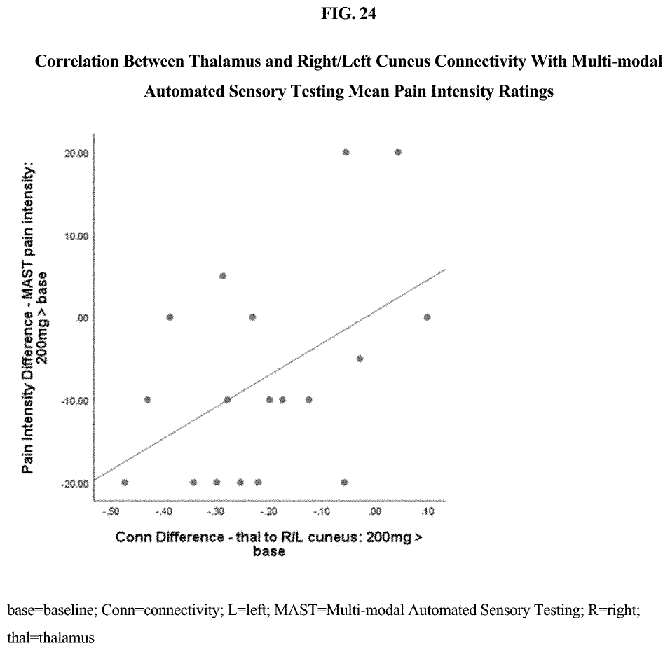

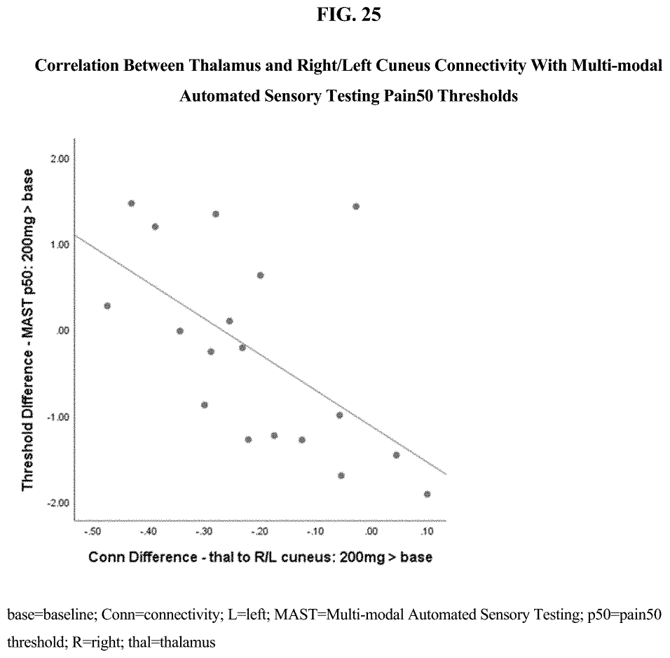

Reduced functional connectivity between the thalamus and bilateral cuneus from the baseline visit to the NYX-2925 200 mg visit are: a) positively correlated with changes in BPI-SF interference scores (r=0.538, p=0.021, see FIG. 23); ie, decreased connectivity between these 2 regions is correlated with improvements in clinical pain interference scores following drug treatment, b) positively associated with the overall pain intensity rating from the MAST testing (r=0.479, p=0.044, see FIG. 24; ie, decreased connectivity between these 2 regions is correlated with reduced pain intensity ratings from MAST testing), and c) negatively associated with pressure thresholds from MAST testing used to elicit a pain rating of 50/100 (r=−0.590, p=0.010, see FIG. 25; ie, decreased connectivity between these 2 regions is associated with increased pressure intensities necessary to evoke moderate pain.

### Quantitative Sensory Testing

### Placebo Effects (Baseline Versus Placebo Visit)

Evoked pain sensitivity was not altered significantly by placebo treatment. However, there was a marginal decrease in pressure pain threshold at the right wrist on placebo compared to baseline (p=0.088), suggesting a placebo-induced increase in pain sensitivity. In contrast, placebo administration produced a marginal decrease in sensitivity to aversive visual stimulation (unpleasantness evoked by the brightest visual stimulus, p=0.055) and cuff-induced pressure pain (Cuff Random Pain50 threshold; p=0.076) compared to baseline.

### NYX-2925 20 mg

Administration of NYX-2925 20 mg produced a small but significant decrease in left (1.60±1.11 versus 1.30±1.08 kgf/cm², p=0.042) and right (1.64±1.30 versus 1.28±1.10 kgf/cm², p=0.021) wrist pressure pain threshold relative to baseline, indicating increased pain sensitivity or hyperalgesia on drug. A marginal decrease in threshold was also observed for MAST Pain50 (p=0.079). In contrast, Cuff Random Pain50 threshold significantly increased with NYX-2925 20 mg (193.00±128.16 mmHg) compared to baseline (151.89±110.64 mmHg, p=0.031), indicating a small analgesic effect of NYX-2925 on deep muscle pressure pain sensitivity. Consistent with this finding, mean pain intensity rating of the Cuff Random paradigm was marginally reduced following drug administration (p=0.073).

There were no significant or marginal effects of NYX-2925 20 mg on QST relative to placebo.

### NYX-2925 200 mg

Compared to baseline, administration of NYX-2925 200 mg resulted in significant decreases in ratings of overall pain intensity (51.43±15.01 vs. 44.76±12.19 NRS units, p=0.035) and pain unpleasantness (50.00±24.24 versus 42.14±19.34, p=0.038) evoked by MAST testing, indicating an analgesic effect of drug administration. Similarly, Cuff Random Pain50 threshold was significantly increased with NYX-2925 200 mg (209.80±132.51 mmHg) compared to baseline (157.70±110.78 mmHg, p=0.038), mirroring the analgesic effect obtained with NYX-2925 20 mg. In contrast, right wrist pressure pain threshold decreased following NYX-2925 200 mg compared to baseline (1.64±1.30 versus 1.32±1.15 kgf/cm², p=0.042), indicating increased pain sensitivity on drug.

Further, right trapezius pressure pain threshold exhibited a small but significant decrease with administration of NYX-2529 200 mg (1.58±1.37) compared to placebo (1.77±1.53 kgf/cm², p=0.047), also suggesting a hyperalgesic action of NXY-2925. There were no additional significant or marginal effects of NYX-2925 200 mg on QST outcomes relative to placebo.

### NYX-2925 20 mg Versus NYX-2925 200 mg

There were no significant or marginal differences between NYX-2925 20 mg and NYX-2925 200 mg on QST.

### NYX-2925 200 mg Versus Week 7 (Follow-Up)

Results showed a decrease in perceived aversiveness of visual stimulation at Week 7 compared to NYX-2925 200 mg. Follow-up ratings of unpleasantness evoked by the brightest visual stimulus (59.97±35.46 versus 53.13±36.88 NRS, p=0.037) and overall visual task unpleasantness (42.31±29.20 versus 33.85±28.07, p=0.040) were decreased relative to NYX-2925 200 mg. In contrast, the cuff algometry procedure was rated slightly more unpleasant overall at Week 7 (30.00±22.82) compared to treatment with NYX-2925 200 mg (25.00±19.15, p=0.040).

### QST Summary

NYX-2925 administration produced somewhat mixed effects on evoked pain sensitivity measured by QST. Pressure pain thresholds measured by hand-held algometry at the wrist and trapezius were decreased following both doses NYX-2925, suggesting a hyperalgesic effect of treatment. Of note, the decrease in trapezius pressure pain threshold following NYX-2925 200 mg was the only significant effect of treatment observed relative to placebo. In contrast to these findings, cuff pain thresholds increased with NYX-2925, indicating an analgesic effect. The cause of this discrepancy between QST measures is unknown but could be related to differences in anatomical targeting (focal versus widespread pressure stimulation) or stimulation paradigms (ascending for algometry versus random for cuff). Regardless, the observed effects are relatively small and likely to be of little clinical relevance. NYX-2925 200 mg treatment also resulted in patients rating the overall MAST procedure as less painful and unpleasant. Interestingly, the actual pain thresholds of the MAST test were not significantly altered by this dose of NYX-2925, suggesting that NYX-2925 may have differential effects on sensory perception thresholds and subjective recall of a painful event. The effects of NYX-2925 on non-painful sensory stimulation remain unclear. Ratings of unpleasantness evoked by visual stimulation decreased after cessation of NYX-2925, suggesting that drug treatment had increased the perceived aversiveness of this task. These results should be interpreted with caution as the QST data available for analysis at follow-up was limited due to its optional designation.

### Analyses of Efficacy

### Numeric Pain Rating Scale Score

A statistically significant mean improvement of −0.44 (p=0.0435, paired t-test) was observed for NPRS average pain score from baseline to end of placebo treatment at Week 2. Mean improvements from Week 2 (placebo) were observed during NYX-2925 treatment at Weeks 3 through 6 and were statistically significant at Week 6. Mean change in NPRS average pain score from Week 2 (placebo) to Week 4 (NYX-2925 20 mg) and Week 6 (NYX-2925 200 mg) was −0.10 and −0.66, respectively. Results were similar for NPRS worst pain score.

In the Per Protocol Population, NPRS average and worst pain scores were similar to those in the Efficacy Population.

### Brief Pain Inventory

The mean changes of −0.50 and −0.26 that were observed for the BPI-SF Pain Severity Index and Pain Interference Index scores, respectively, from baseline to end of placebo treatment at Week 2 were not statistically significant. The mean changes from Week 2 (placebo) that were observed for both scores during NYX-2925 treatment at Weeks 4 and 6 were not statistically significant. Mean change in the BPI-SF Pain Severity Index score from Week 2 (placebo) to Week 4 (NYX-2925 20 mg) and Week 6 (NYX-2925 200 mg) was −0.22 and −0.27, respectively. Mean change in the Pain Interference Index score from Week 2 (placebo) to Week 4 (NYX-2925 20 mg) and Week 6 (NYX-2925 200 mg) was −0.04 and −0.66, respectively.

In the Per Protocol Population, BPI-SF Pain Severity Index and Pain Interference Index scores were similar to those in the Efficacy Population.

### Patient-Reported Outcomes Measurement Information System Fibromyalgia Fatigue Profile

The mean change of 0.2 that was observed for PROMIS_{FM} Fatigue Profile Total Score from baseline to end of placebo treatment at Week 2 was not statistically significant. The mean change from Week 2 (placebo) to Week 4 (NYX-2925 20 mg) was not statistically significant. A statistically significant mean decrease (improvement) from Week 2 (placebo) was observed at Week 6 (NYX-2925 200 mg). Mean change in PROMIS_{FM} Fatigue Profile Total Score from Week 2 (placebo) to Week 4 (NYX-2925 20 mg) and Week 6 (NYX-2925 200 mg) was −3.4 and −5.6, respectively.

Statistically significant mean decreases (improvements) of −2.2, −1.4, and −1.0 were also observed for the PROMIS_{FM} Experience, Social Impact, and Motivational Impact Subdomain Scores, respectively, from Week 2 (placebo) to Week 6 (NYX-2925 200 mg). The changes from Week 2 (placebo) that were observed for the PROMIS_{FM} Cognitive Impact Subdomain Score during NYX-2925 treatment at Weeks 4 and 6 were not statistically significant.

In the Per Protocol Population, Total PROMIS_{FM} fatigue profile scores were similar to those in the Efficacy Population.

### Multidimensional Inventory of Subject Cognitive Impairment

The mean change of 1.4 that was observed for the Multidimensional Inventory of Subject Cognitive Impairment Total scores from baseline to end of placebo treatment at Week 2 was not statistically significant. The mean improvements from Week 2 (placebo) that were observed during NYX-2925 treatment at Weeks 4 and 6 were not statistically significant. Mean change in the total score from Week 2 (placebo) to Week 4 (NYX-2925 20 mg) and Week 6 (NYX-2925 200 mg) was 1.1 and 0.9, respectively.

In the Per Protocol Population, Multidimensional Inventory of Subject Cognitive Impairment Total scores were similar to those in the Efficacy Population.

### Revised Fibromyalgia Impact Questionnaire

The mean changes of −2.2, −1.6, −1.1, and −2.9 that were observed for the Revised Fibromyalgia Impact Questionnaire Function Domain, Overall Impact Domain, Symptoms Domain, and Total scores, respectively, from baseline to end of placebo treatment at Week 2 were not statistically significant. Mean improvements from Week 2 (placebo) were observed for each domain/total score during NYX-2925 200 mg treatment at Week 6, with statistically significant changes for the Revised Fibromyalgia Impact Questionnaire Symptoms Domain and Total scores. Mean improvements were as follows:
Mean change in the Function Domain score from Week 2 (placebo) to Week 6 (NYX-2925 200 mg) was −3.7.
Mean change in the Overall Impact Domain score from Week 2 (placebo) to Week 6 (NYX-2925 200 mg) was −2.0.
Mean change in the Symptoms Domain score from Week 2 (placebo) to Week 4 (NYX-2925 20 mg) and Week 6 (NYX-2925 200 mg) was −2.1 and −6.2, respectively.
Mean change in the Total score from Week 2 (placebo) to Week 4 (NYX-2925 20 mg) and Week 6 (NYX-2925 200 mg) was −0.5 and −6.3, respectively.

In the Per Protocol Population, Revised Fibromyalgia Impact Questionnaire Function Domain, Overall Impact Domain, Symptoms Domain, and Total scores were similar to those in the Efficacy Population.

Patient-Reported Outcomes Measurement Information System Sleep Disturbance—Short Form

The mean change of −1.4 that was observed for the PROMIS Sleep Disturbance—Short Form, from baseline to end of placebo treatment at Week 2 was not statistically significant. The changes from Week 2 (placebo) that were observed during NYX-2925 treatment at Weeks 4 and 6 were not statistically significant. The mean change in the PROMIS Sleep Disturbance—short form score from Week 2 (placebo) to Week 4 (NYX-2925 20 mg) and Week 6 (NYX-2925 200 mg) was −0.9 and −1.6, respectively.

In the Per Protocol Population, PROMIS Sleep Disturbance—short form scores were similar to those in the Efficacy Population.

Hospital Anxiety and Depression Scale

A mean change of −0.3 from baseline to end of placebo treatment at Week 2 was observed for the Total Anxiety score and no mean change was observed for the Total Depression score. No statistically significant mean changes from Week 2 (placebo) were observed for the Total Anxiety and Total Depression scores at Weeks 4 and 6.

In the Per Protocol Population, the Hospital Anxiety and Depression Scale Total Anxiety and Total Depression scores were similar to those in the Efficacy Population.

Patient-Reported Outcomes Measurement Information System Physical Function—Short Form

The mean changes of 0.1 and 0.2 that were observed for the PROMIS Physical Function—Short Form Walking and Other Physical Function Subdomains, from baseline to end of placebo treatment at Week 2 were not statistically significant. The changes from Week 2 (placebo) that were observed during NYX-2925 treatment at Week 6 were not statistically significant. Mean change in the PROMIS Physical Function—Walking Subdomain scores from Week 2 (placebo) to Week 4 (NYX-2925 20 mg) and Week 6 (NYX-2925 200 mg) was 0.2 and 0.5, respectively. The mean change in the PROMIS Physical Function—Other Physical Function Subdomain scores from Week 2 (placebo) to Week 4 (NYX-2925 20 mg) and Week 6 (NYX-2925 200 mg) was −0.1 and 0.4, respectively.

In the Per Protocol Population, PROMIS Sleep Disturbance—short form scores were similar to those in the Efficacy Population.

Patient Global Impression of Change

Scores for the Patient Global Impression of Change indicated little, if any, change in severity during the study when the traditional definition of “improved” comprising only the categories of “very much improved” and “much improved” was considered. However, due to the short duration of exposure at both the 20 mg and 200 mg dose levels, an additional analysis defining “improved” as comprising “very much improved,” “much improved”, and “minimally improved” was evaluated post hoc. With this categorization, a larger proportion of subjects had a reduction of severity in Patient Global Impression of Change on the 20 mg and 200 mg treatment conditions versus placebo.

In the Per Protocol Population, the Patient Global Impression of Change was similar to that in the Efficacy Population.

Fibromyalgia Survey Questionnaire

The mean changes of −1.0 and −0.4 that were observed for the Widespread Pain Index and Symptom Severity score from baseline to end of placebo treatment at Week 2 were not statistically significant. The mean changes from Week 2 (placebo) that were observed at Week 4 and Week 6 for the Widespread Pain Index were not statistically significant. Mean change in the Widespread Pain Index from Week 2 (placebo) to Week 4 (NYX-2925 20 mg) and Week 6 (NYX-2925 200 mg) was 0.0 and −1.3, respectively.

The mean change from Week 2 (placebo) for the Symptom Severity score was statistically significant at Week 6 but was not statistically significant at Week 4. Mean change in the Symptom Severity score from Week 2 (placebo) to Week 4 (NYX-2925 20 mg) and Week 6 (NYX-2925 200 mg) was −0.3 and −0.9, respectively.

Pharmacodynamic Conclusions

Multiple effects of NYX-2925 were observed on Glx levels acquired during ¹H-MRS. NYX-2925 reduced Glx levels in the dACC at rest and the posterior insula following experimental pressure pain. Higher pre-treatment Glx levels were also associated with subsequent increases in pressure pain thresholds following NYX-2925 20 mg. The relationships between Glx and QST-derived pain thresholds were opposite during NYX-2925 200 mg as compared to pre-treatment timepoints.

NYX-2925 also had effects on brain connectivity patterns, showing decreases in connectivity to specific regions in the dACC, pgACC, thalamus, and medial prefrontal cortex. Of note some of the regional brain areas showing decreased connectivity, also showed effects of NYX-2925 on Glx. One region in particular was the dACC. This region displayed marked decreases in connectivity to the precuneus (a region of the Default Mode Network) and this change in connectivity was associated with decreases in pain interference. These findings suggest that NYX-2925 induces changes in brain functional connectivity.

Efficacy Conclusions

Statistically significant improvements were observed from Week 2 (placebo) to Week 6 (NYX-2925 200 mg) for the following efficacy endpoints:
NPRS average pain score, with mean change of −0.66 (p=0.0072, paired t-test)
NPRS worst pain score, with mean change of −0.61 (p=0.0360, paired t-test)
PROMIS_{FM} Total Score, with mean change of −5.6 (p=0.0049, paired t-test)
PROMIS_{FM} Experience Subdomain Score, mean change of −2.2 (p=0.0036, paired t-test)
PROMIS_{FM} Social Impact Subdomain Score, with mean change of −1.4 (p=0.0329, paired t-test)
PROMIS_{FM} Motivational Impact Subdomain Score, with mean change of −1.0 (p=0.0490, paired t-test)
Revised Fibromyalgia Impact Questionnaire Symptoms Domain, with mean change of −6.2 (p=0.0207, paired t-test)
Revised Fibromyalgia Impact Questionnaire Total score, with mean change of −6.3 (p=0.0135, paired t-test)

Mean reductions in the BPI-SF Pain Severity Index and Pain Interference Index scores from Week 2 (placebo) to Week 6 (NYX-2925 200 mg) were −0.27 and −0.66, respectively. A mean improvement was also observed for the Multidimensional Inventory of Subject Cognitive Impairment Total score of 0.9 from Week 2 (placebo) to Week 6 (NYX-2925 200 mg). These findings were not statistically significant.

Smaller mean improvements from Week 2 (placebo) were generally observed following treatment with NYX-2925 20 mg (Week 4); however, trends were positive for the treatment of fibromyalgia with NYX-2925 at a 20 mg dose.

In summary, consistent improvement following treatment with NYX-2925 was observed across measures of pain and quality of life in subjects with fibromyalgia.

### INCORPORATION BY REFERENCE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating fibromyalgia in a patient in need thereof, comprising administering daily to the patient an amount of (2S, 3R)-3-hydroxy-2-((R)-5-isobutyryl-1-oxo-2,5-diazaspiro[3.4]octan-2-yl)butanamide (“NYX-2925”), or a pharmaceutically acceptable salt thereof, wherein the amount is between about 20 mg and about 200 mg.

2. The method of claim 1, wherein the amount is between about 25 mg to about 180 mg, between about 25 mg to about 160 mg, between about 30 mg to about 140 mg, between about 35 mg to about 120 mg, between about 40 mg to about 100 mg, between about 45 mg to about 80 mg, or between about 50 mg to about 60 mg.

3. The method of claim 1, wherein the amount is between about 44 mg to about 56 mg, between about 45 mg to about 55 mg, between about 46 mg to about 54 mg, between about 47 mg to about 53 mg, between about 48 mg to about 52 mg, or between about 49 mg to about 51 mg.

4. The method of claim 1, wherein the amount is about 20 mg, about 50 mg, about 100 mg, or about 200 mg.

5. The method of claim 1, wherein, after daily administration of NYX-2925, or a pharmaceutically acceptable salt thereof, the patient has a reduction in levels of resting Glx in the dorsal anterior cingulate cortex after an evoking pain stimulus, wherein the level of Glx is the sum of a glutamate level and a glutamine level in the patient.

6. The method of claim 1, wherein, after daily administration of NYX-2925, or a pharmaceutically acceptable salt thereof, the patient has a reduction in the increase of levels of Glx in the posterior insular cortex after an evoking pain stimulus, wherein the level of Glx is the sum of a glutamate level and a glutamine level in the patient.

7. The method of claim 1, wherein the patient has reduced levels of Glx in key pain-regulating brain regions after daily administration of NYX-2925, or a pharmaceutically acceptable salt thereof, wherein the level of Glx is the sum of a glutamate level and a glutamine level in the patient.

8. The method of claim 1, wherein, after daily administration of NYX-2925, or a pharmaceutically acceptable salt thereof, the patient has a reduced average daily pain score.

9. The method of claim 1, wherein after daily administration of NYX-2925, or a pharmaceutically acceptable salt thereof, the patient has a reduced worst daily pain score.

10. The method of claim 1, wherein after daily administration of NYX-2925, or a pharmaceutically acceptable salt thereof, the patient has a reduced Revised Fibromyalgia Impact Questionnaire (“FIQR”) score.

11. The method of claim 1, wherein, after daily administration of NYX-2925, or a pharmaceutically acceptable salt thereof, the patient has a reduced PROMIS_{FM} Fatigue Profile total score.

12. The method of claim 1, wherein administering comprises administering daily to the patient a pharmaceutical formulation, wherein the pharmaceutical formulation comprises:
NYX-2925, or a pharmaceutically acceptable salt thereof, present in an effective amount;
microcrystalline cellulose;
pregelatinized starch, and
magnesium stearate.

13. The method of claim 12, wherein the pharmaceutical formulation is encapsulated in a capsule.

14. The method of claim 13, wherein the capsule comprises hydroxyl-propyl cellulose.

15. The method of claim 12, wherein the capsule comprises about 10 mg of NYX-2925, or a pharmaceutically acceptable salt thereof; about 50 mg of NYX-2925, or a pharmaceutically acceptable salt thereof; about 100 mg NYX-2925, or a pharmaceutically acceptable salt thereof, or about 200 mg NYX-2925, or a pharmaceutically acceptable salt thereof.

16. The method of claim 12, wherein the pharmaceutical formulation is in the form of a tablet.

17. The method of claim 16, wherein the tablet comprises about 10 mg of NYX-2925, or a pharmaceutically acceptable salt thereof; about 50 mg of NYX-2925, or a pharmaceutically acceptable salt thereof; about 100 mg NYX-2925, or a pharmaceutically acceptable salt thereof, or about 200 mg NYX-2925, or a pharmaceutically acceptable salt thereof.

18. The method of claim 1, wherein the patient is human.

19. The method of claim 1, wherein administering is administering orally.

20. A capsule comprising a pharmaceutical formulation, the pharmaceutical formulation comprising about 10 mg of NYX-2925, or a pharmaceutically acceptable salt thereof; about 50 mg of NYX-2925, or a pharmaceutically acceptable salt thereof; about 100 mg NYX-2925, or a pharmaceutically acceptable salt thereof; or about 200 mg NYX-2925, or a pharmaceutically acceptable salt thereof; and a pharmaceutically acceptable excipient.
